(19) United States
(12) Patent Application Publication
(10) Pub. No.: US 2004/0253243 A1
(43) Pub. Date: Dec. 16, 2004

(54) Aptamer therapeutics useful in ocular pharmacotherapy

(76) Inventors: David Epstein, Belmont, MA (US); Dilara Grate, Waltham, MA (US); Jeffrey Kurz, Arlington, MA (US); Nicholas Marsh, Boston, MA (US); Thomas Greene McCauley, Cambridge, MA (US); Charles Wilson, Concord, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/762,915

(22) Filed: Jan. 21, 2004

Related U.S. Application Data
(60) Provisional application No. 60/441,357, filed on Jan. 21, 2003.
(60) Provisional application No. 60/463,095, filed on Apr. 15, 2003.
(60) Provisional application No. 60/464,179, filed on Apr. 21, 2003.
(60) Provisional application No. 60/465,055, filed on Apr. 23, 2003.
(60) Provisional application No. 60/491,019, filed on Jul. 29, 2003.
(60) Provisional application No. 60/512,071, filed on Oct. 17, 2003.
(60) Provisional application No. 60/469,628, filed on May 8, 2003.
(60) Provisional application No. 60/474,680, filed on May 29, 2003.
(60) Provisional application No. 60/537,045, filed on Jan. 16, 2004.
(60) Provisional application No. 60/537,201, filed on Jan. 16, 2004.

(51) Int. Cl. A61K 39/395

(52) U.S. Cl. 424/145.1

(57) ABSTRACT

The invention provides nucleic acid therapeutic compositions capable of binding to cytokines, growth factors and cell surface proteins, individually or in combinations of two or more, and methods for delivering these nucleic acid therapeutics in the treatment of glaucoma and other proliferative diseases of the eye.

### PREFERENCE TO RELATED APPLICATIONS

This application claims priority under 35 U.S.C. § 119(e) to the following provisional applications: U.S. Provisional Application Ser. No. 60/441,357, filed Jan. 21, 2003; U.S. Provisional Application Ser. No. 60/463,095, filed Apr. 15, 2003; U.S. Provisional Application Ser. No. 60/464,179, filed Apr. 21, 2003; U.S. Provisional Application Ser. No. 60/465,055, filed Apr. 23, 2003; U.S. Provisional Application Ser. No. 60/491,019, filed Jul. 29, 2003; U.S. Provisional Application Ser. No. 60/512,071, filed Oct. 17, 2003; U.S. Provisional Application Ser. No. 60/469,628, filed May 8, 2003; U.S. Provisional Application Ser. No. 60/474,680, filed May 29, 2003; U.S. Provisional Application Ser. No. 60/______ , filed on Jan. 16, 2004; and U.S. Provisional Application Ser. No. 60/______ , filed on Jan. 16, 2004, each of which is herein incorporated by reference in its entirety.

### FIELD OF THE INVENTION

The invention relates generally to the field of nucleic acid therapeutics and more particularly to nucleic acid therapeutic compositions capable of binding to cytokines, growth factors and cell surface receptors, individually or in combinations of two or more, and methods for delivering these nucleic acid therapeutics in the treatment of glaucoma and other proliferative diseases of the eye.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (MAbs), are capable of specifically binding to selected targets and, through binding, block their targets' ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (e.g., hydrogen bonding, electrostatic complementarity, hydrophobic contacts, and steric exclusion) that drive affinity and specificity in antibody-antigen complexes.

Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial therapeutic leads. In vitro selection allows the specificity and affinity of the aptamer to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC, and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus, large volumes necessary for most therapeutic MAbs. With good solubility (>150 mg/ml) and comparatively low molecular weight (aptamer: 10-50 kDa; antibody: 150 kDa), a weekly dose of aptamer may be delivered by injection in a volume of less than 0.5 ml. Aptamer bioavailability via subcutaneous administration is >80% in monkey studies (Tucker et al., J. Chromatography B. 732: 203-212, 1999). In addition, the small size of aptamers allows them to penetrate into areas of conformational constrictions that do not allow antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics, and the capital cost of a large-scale protein production plant is enormous, a single large-scale synthesizer can produce upwards of 100 kg of oligonucleotide per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to factors such as heat and denaturants, and can be stored for extended periods (>1 yr) at room temperature as lyophilized powders. In contrast, antibodies must be stored in a refrigerated environment.

Glaucoma

Two leading causes of vision loss are glaucoma and age-related maculodegenerative disease (AMD). Glaucoma is a proliferative disease of the eye affecting 2.2 million patients in the U.S. and 65 million patients worldwide. Glaucoma disease is associated with reduced fluid drainage from the eye and an elevation in intraocular pressure (IOP). When IOP is high, individual nerve fiber cells die leading to vision loss. Vision loss is manifested by characteristic optic disc damage, nerve fiber layer defects, visual field loss starting at the periphery, and eventual blindness. Glaucoma disease progression is currently irreversible, but it may be slowed with therapeutic drugs to modulate fluid production and IOP. The current therapeutic agents of choice in treating advanced glaucoma are cytotoxic agents delivered by trabeculectomy. There are estimates of approximately 120,000 surgeries each year to treat glaucoma patients in the U.S.

The first line of glaucoma treatment is typically the use of therapeutic drugs to modulate intraocular fluid levels. Glaucoma filtering microsurgery, or trabeculectomy, is a second line of treatment in which a tiny puncture is made in the sclera of the eye to allow fluid to drain into a bleb, thereby reducing IOP. However, post-surgical complications are significant and can lead to continued vision loss. Complications from surgery arise when incomplete wound healing and scarring results in a return to high IOP and a need for additional surgery. During trabeculectomy, antibiotics and corticosteroids can be injected subjunctivally into the inferior fornix or collagen shields soaked in them can be used to cover the eye to control the extent of post-surgical scarring. In order to combat the effects of scarring, antimetabolite agents, such as mitomycin-C and 5-fluorouracil, are used to control the extent of post-surgical scarring. A failed trabeculectomy is considered to exist when there is less than a 25% drop in IOP post-surgically, in which case a second drainage surgery is performed.

To prevent trabeculectomy failure, topical steroids and or antifibrotic agents are commonly used. Steroids like prednislolone acetate 1%, 4-6 times daily are often used postoperatively and tapered after 4-8 weeks. Cycloplegics such as atropine 1% or cyclopentolate 1% can be sued up to four times daily in cases prone to shallow anterior chamber. To prevent excessive postoperative subconjunctival fibrosis, adjunctive antimetabolites such as mitomycin C and 5-fluorouracil are used. They inhibit fibroblast proliferation and subsequent scar tissue formation. Mitomycin C is 100 times more potent than 5-FU. Both are associated with a higher success rate but also with a higher complication rate so use is determined on a case by case basis. Mitomycin C (0.2-0.5mg/ml solution) or 5-FU (25-50 mg/ml solution) can be applied for 1-5 minutes using a soaked cellulose sponge or filter paper over the episclera before dissecting the scleral flap. They can also be applied under the scleral flap and time of exposure may vary depending on the expected risk, of fibrosis. Further, during surgery, the conjunctival-Tenon's layer is draped over the sponge avoiding contact with the wound edge. After removal, the entire area is cleansed thoroughly with a salt solution. 5-FU can be delivered subconjunctivally in 5 mg aliquots. Total number of injections is adjusted depending on filtering bleb function and tolerance of the corneal epithelium. Complications associated with 5-FU include corneal and conjunctival epithelial toxicity, corneal ulcers, conjunctival wound leaks, subconjunctival hemorrhage or inadvertent intraocular spread of 5-FU.

Severe complications can arise from repeat trabeculectomies and the use of antimetabolite therapy, including fluid leakage, intraocular hypotony (low IOP), and general tissue toxicity (Blindish, et al., Ophthalmology (2002), 109:1336-1341; Belyea, et al, Am J Ophthalmol (1999), 124:40-45; Kupin, et al., Am J Ophthalmol (1995), 119:30-39). Antimetabolite agents can further damage eye tissue leading to low IOP, or even blindness. Failure of antimetabolite therapy in glaucoma treatment is defined by a two-line drop in Snellen visual acuity tests (Membrey, et al., (2000). Br J Ophthalmology, 84:1154-58).

Progression of glaucoma disease is associated with increases in the level(s) of transforming growth factor cytokines in the eye. The transforming growth factor β (TGFβ subfamily) is comprised of three members TGFβ1, TGFβ2, and TFGβ3. TGFβs are multifunctional cytokines that control growth, differentiation, and development. They are expressed by many different cell types, and most cells are responsive to TGFβs. Transforming growth factor beta 2 (TGFβ2) is a 25 kD homodimer growth factor cytokine that is involved in cell proliferation, differentiation, and extracellular matrix formation. Several receptors (types I-V) mediate the cellular response to TGFβ2 and its isoforms TGFβ-1 and TGFβ-3 in a variety of cells. The type II receptor is the main signaling receptor responsive to TGFβ2, although high affinity binding of TGFβ2 to the type III, non-signaling receptor is believed to enhance type II dependent signaling.

In many parts of the body, TGFβ1 predominates, but in the eye, TGFβ2 is the predominant form. Transforming growth factor beta 2 (TGFβ2) is implicated in ocular wound healing and is suggested to play a role in scarring associated with glaucoma surgery. The ocular scarring response is mediated by TGFβ2 (Cordeiro, et al., Invest Ophthalmol. Vis. Sci. (1999), 40:1975-1982). Elevated levels of TGFβ2 are detected in aqueous humor in glaucomatous eyes compared to control levels in normal eyes: 21 pM glaucomatous versus 12 pM for normal eyes (Ochiai, et al., Jpn J. Ophthalmol. (2002), 46:249-253). Trabecular meshwork cells, as well as ciliary body cells, express and secrete TGFβ2. It has been suggested that TGFP2 contributes to the excess accumulation of extracellular components in the aqueous outflow system observed in aging and glaucomatous eyes (Tripathi, et al., Exp. Eye Res. (1994), 58:523-528).

There are clinical trials currently underway testing the use of alternative therapeutics, such as antibodies specific to TGFβ2, which show prevention of excessive post-operative scarring in patients undergoing eye surgery for glaucoma (Broadway, et al., Adjunctive anti-TGFβ2 human MAb as a novel agent to prevent scarring following phacotrabeculectomy. May 2002, ARVO Meeting Poster #3331). However, there are undesired side effects from antibody therapy in the eye, such as inflammation or immune responses to the foreign antibody, which can lead to secondary causes of increase in IOP in the glaucomatous eye. There have also been studies with anti-sense nucleic acids that inhibit expression of TGFβ2 in the eye leading to reduced surgical scarring and improved surgical outcome in the rabbit model (Cordeiro, et al., Gene Therapy (2003), 10:59-71). However, this approach may interfere with normal, post-surgical healing and tissue regeneration processes in the eye and contribute to cytotoxicity within the eye and surrounding tissue. Thus, the use of these alternative therapeutics has not completely eliminated the side effects and secondary deleterious effects of the current glaucoma therapeutics.

Age-Related Maculodegenerative Disease (AMD)

Age-Related Maculodegenerative Disease (AMD) is a degenerative condition of the macula. It is the most common cause of vision loss in the United States in those 50 years old or older, and its prevalence increases with age. It affects 15 million people in the United States alone. AMD is caused by hardening of the arteries that nourish the retina. This deprives the retinal tissue of oxygen and nutrients that it needs to function and thrive. As a result, the central vision deteriorates. AMD is classified as either wet (neovascular) or dry (non-neovascular), based on the absence or the presence of abnormal growth of blood vessels under the retina.

Wet AMD affects about 10% of patients who suffer from macular degeneration. This type occurs when new vessels form to improve the blood supply to oxygen-deprived retinal tissue. However, the new vessels are very delicate and break easily, causing bleeding and damage to surrounding tissue. The wet form can manifest in two types: classic or occult. Over 70% of patients with the wet form have the occult type. So far, only the classic wet type is treated with conventional laser photocoagulation to stabilize vision or to limit the growth of abnormal blood vessels. The remaining majority of patients with wet AMD cannot be treated with the laser procedure. The current laser treatment does not improve vision in most treated eyes because the laser destroys not only the abnormal blood vessel but also the overlying macula.

Dry AMD although more common, typically results in a less severe, more gradual loss of vision. It is characterized by drusen and loss of pigment in the retina. Drusen are small, yellowish deposits that form within the layers of the retina. Currently there is no proven treatment for the dry type, but the loss of vision tends to be milder and the disease progression is rather slow. There is no currently proven medical therapy for dry macular degeneration.

Proliferative Vitreo-Retinopathy (PVR)

Other causes of blindness are retinal detachments. Retinal detachments have an annual incidence in the general population of 1:10,000. There are, however, a variety of associated ocular and systemic disease states that increase the chances of retinal detachment. These include: diabetes, high myopia, pseudophakia and aphakia, blunt and penetrating ocular trauma, and cytomegalovirus retinitis associated with acquired immunodeficiency syndrome. Vitrectomy is the standard of care for retinal detachment. Annually, there are about 200,000 vitrectomies in the U.S., and 300,000 vitrectomies outside the United States. Proliferative Vitroretinopathy (PVR) occurs in ˜10% of retinal detachments or 62,600 cases a year worldwide and ˜2,800 cases a year in the U.S. PVR is the most common cause of failure in retinal reattachment surgeries.

Platelet derived growth factor (PDGF) is a strong mitogen and is known to play a crucial role in a variety of proliferative diseases. PDGF is postulated to be involved in the regulation of abnormal growth and migration of glial and retinal pigmented endothelial cells (RPE) cells in PVR.

PDGF forms dimers of the A and B subunits, i.e., AB heterodimers, and AA and BB homodimers. PDGF has a pivotal role in regulation of normal cell proliferation and the mediation of pathological cell growth such as tissue, fibrosis, proliferative disorders and angiogenesis. It is involved in restenosis, renal scarring, wound healing and cancer. Most tumor cell lines secrete PDGF, and heavily express PDGF receptor (PDGF-R). The amino acid sequence of PDGF resembles that of an oncogene.

High retinal expression of PDGF results in traction retinal detachment from proliferation of both vascular and nonvascular cells. PDGF promotes proliferation of trabecular meshwork cells, enhances dedifferentiation of retinal pigment epithelial from hexagonal to flattened cells, increases expression of alpha smooth muscle actin, enhances myoid differentiation and collagen gel contraction. There are elevated levels in the vitreous of patients with AMD.

While many growth factors are thought to contribute to PVR, including TGFβ, VEGF, BFGF, HGF and IL-6P, PDGF has been shown to play the most important role.

PVR is the most common complication following a retinal detachment associated with a retinal hole or break. PVR refers to the growth of cellular membranes (composed primarily of glial cells and retinal pigment epithelial cells, but also fibroblasts and inflammatory cells) within the vitreous cavity and on the front and back surfaces of the retina. These membranes, which are essentially scar tissues, exert traction on the retina and may result in recurrences of retinal detachment, even after an initially successful retinal detachment procedure. PVR may be associated with spontaneous reopening of otherwise successfully treated retinal breaks and may even cause the development of new retinal breaks. It may be associated with severe distortion and “stiffness” of the retina, as a result of the contracting membranes. This aspect of the condition results in damage to vision.

PDGF promotes proliferation of trabecular meshwork cells, enhances dedifferentiation of retinal pigment epithelial from hexagonal to flattened cells, increases expression of alpha smooth muscle actin, enhances myoid differentiation and collagen gel contraction. There are elevated levels in the vitreous and preretinal membranes of patients with PVR. In experimental models, cells without PDGFRs were ineffective in inducing PVR. In particular, it is PDGF-AA that results in extensive proliferation of glial cells and traction retinal detachment without vascular cell involvement. It has been shown that it is the PDGF-alphaR that is capable of driving the events leading to PVR. PDGF mutants are all capable of blocking PDGF stimulated cell cycle progression and varied in their kinase activity and ability to block PVR. A truncated receptor is effective in blocking PVR.

The stages of PVR development are: 1)Breakdown of the blood-retinal barrier; 2)Chemotaxis and cellular migration; 3)Cellular proliferation; 4)Membrane formation with remodeling of the extracellular matrix; and 5)Contraction. PDGF plays an important role at each of these stages as described below for each of the five stages of PVR development.

The RPE forms a mosaic of cells between the choroid and neural retina that serves as the outer blood-retinal barrier regulating retinal homeostasis and visual function. The initial step in PVR is the dedifferentiation of RPE cells: morphological alteration from a mitotically quiescent hexagonal shape to a migrating flattened shape with a loss of epithelial characteristics. Additionally, RPE cells decrease their expression of cytokeratin and begin expression of alpha smooth muscle actin (alpha-SMA). Alpha-SMA is essential for contractile activity and it increases in a time dependent manner. PDGF enhances dedifferentiation of RPE cells, myoid differentiation and alpha-SMA expression.

The breakdown of the blood-retinal barrier allows for entry of various cell types including RPE cells, glial cells, fibroblasts, macrophages, leucocytes and serum components into the vitreous and subretinal space. RPE and glial cells are the predominant types. These cells adhere to the retina and vitreous gel. PDGF is a potent stimulator of RPE and glial cell migration.

Once attached to the retina and vitreous gel these cells proliferate extensively. PDGF triggers proliferation of RPE and glial cells and induces DNA synthesis.

RPE cells transdifferentiate to myofibroblasts or mesenchymal-like cells and form epiretinal membranes on the surface of the retina and within the vitreous and begin to synthesize extracellular matrix. Normal RPE cells do not express PDGF or its receptor. However, PDGF and it's receptor are highly expressed on RPE cells that form PVR membranes. PDGF stimulates fibroblasts to synthesize and deposit collagen. Finally, the membranes exert contractile force and traction on the attached retina leading to reopening of breaks and to retinal detachment. PDGF potentiates RPE contractile ability and stimulates fibroblast and collagen gel contraction.

Proliferative Diabetic Retinopathy (PDR)

Proliferative Diabetic Retinopathy (PDR) is a complication of diabetes that is caused by changes in the blood vessels of the retina. When blood vessels in the retina are damaged, they may leak blood and grow fragile, brush-like branches and scar tissue. This can blur or distort the vision images that the retina sends to the brain. Diabetic retinopathy is a major cause of blindness in developed countries and is the leading cause in diabetic patients aged 25 to 74. It is responsible for 12,000 to 24,000 new cases of blindness in the U.S. each year. It is estimated that 25% of diabetics suffer from diabetic retinopathy and incidence increases to 60% after 5 years and 80% after 10-15 years with type I diabetes. The U.S. patient population is 5 million and the potential U.S. market is $5 billion. The disease is characterized by hyperglycaemia, basement membrane thickening, pericyte loss, microaneurysms and preretinal neovascularization which can lead to blindness through hemorrhage and tractional retinal detachment.

Nonproliferative diabetic retinopathy is characterized by intraretinal microaneurysms, hemorrhages, nerve-fiber-layer infarcts, hard exudates and microvascular abnormalities. Macular edema is the principle mechanism for vision loss. It results from vascular leakage from microaneurysms in the macular (central area of the retina) capillaries. Leakage may progress to macular thickening associated with hard exudates or cystoid changes and this often results in various degrees of central vision loss. Proliferative diabetic retinopathy is characterized by retinal neovascularization. It is graded according to the presence, location, severity and associated hemorrhagic activity of retinal neovascularization. It is associated with severe vision loss. The pathology of diabetic retinopathy can be attributed to the following disease states. Circulation problems cause regions of the retina to become oxygen deprived or ischemic. Neo vascularization causes new vessels to start to grow within the vitreous to maintain adequate oxygen levels. Blood seeping out of the newly formed capillaries and the formation of scar tissue creates traction on the retina causing small tears. Tears are followed by fluid build-up underneath or in between the layers of the retina and detachment occurs. Patients experience blurred vision, floaters, flashes and sudden loss of vision due to the hemorrhaging, edema and scar tissue formation.

Vitrectomy is a microsurgical procedure used to repair retinal disorders, many of which were previously considered inoperable. Victrectomy is 90% successful if performed before the retina is seriously damaged.

PDGF-B plays an important role in the pathogenesis of PDR in synergistic action with other growth factors. Hypoxia increases expression of PDGF-B. High retinal expression of PDGF-B results in traction retinal detachment from proliferation of both vascular and nonvascular cells. PDGF-B induces proliferation of several cell types in the retina including astrocytes, pericytes, and endothelial cells. The cells proliferate on the surface of the retina and cords of cells migrate into the inner nuclear layer and exert traction on the retina, resulting in outer retinal folds and focal areas of detachment that enlarge and lead to total retinal detachment.

This feature of PDR membranes makes treatment difficult and generally requires cutting connections within the retina for removal rather than just peeling the membranes from the surface. PDGF directly acts on endothelial cells through PDGFR-b to induce angiogenesis. PDGF has a pivotal role in regulation of normal cell proliferation and the mediation of pathological cell growth such as tissue, fibrosis, proliferative disorders and angiogenesis. It is involved in restenosis, renal scarring, wound healing and cancer. PDGF acts on fibroblasts, smooth muscle cells, neuroglial cells and stimulates proliferation of connective-tissue cells.

Other Growth Factors Cytokines and Cell Surface Proteins

Other growth factors, cytokines and cell surface proteins are implicated in ocular wound healing and are suggested to play a role in scarring associated with glaucoma surgery. Such cytokines, cell surface proteins and growth factors implicated in various ocular diseases include ICAM-1, IGF-1, VEG/JVEGF-R, TNF-α, and αVβ3. Intercellular Adhesion Molecule 1 (ICAM-1) is a 76 to 115 kDa surface glycoprotein with five extracellular immunoglobulin-like domains that plays a particularly important role in diabetic retinopathy. ICAM-1-mediated leukostasis is causative in the pathogenesis of diabetic retinopathy. ICAM-1 interactions with β2 integrins located on the surface of leukocytes (neutrophils, basophils, lymphocytes, eosinophils, monocytes) are important for their firm adhesion to the endothelium and their transendothelial migration to sites of inflammation. ICAM-1 facilitates adhesion of leukocytes to the retinal vasculature in diabetic retinopathy and is involved with retinal endothelial cell injury and death via lesions that produce irreversible retinal ischemia through inability of capillaries to support blood flow. Inhibition of ICAM-1 bioactivity blocks diabetic retinal leukostasis and potently prevents blood-retinal barrier breakdown.

Insulin-like Growth Factor-1 (IGF-1) is a 7.5 kDa peptide, having 50% homology to proinsulin (50%) and is produced primarily in the liver under control of growth hormone. IGF-1 is a potent mitogen/stimulator of cell proliferation and a strong anti-apoptotic agent. Its function is modulated by six IGF binding proteins (IGFBPs), and its levels are influenced by developmental stage and nutrition. The effects of IFG-1 range from cell growth and protection, resistance to oxidative stress, promotion of bone and muscle growth, and protection of neuronal cells. IGF-1 is implicated in angiogenesis, and IGF-1 in conjunction with VEGF has been implicated in playing a role in proliferative diabetic retinopathy (PDR). PDR is a complication of diabetes that is caused by changes in the blood vessels in the retina. When blood vessels in the retina are damaged, they may leak blood and grow fragile, brush-like branches and scar tissue, which can blur or distort vision images that the retina sends to the brain.

Transcription of vascular endothelial growth factor and its receptor (VEGF and VEGF/R) is enhanced by advanced glycation end products and by the presence of insulin. The accumulation of advanced glycation end products in the diabetic retina contributes to neovascularization, which can result in loss of vision. The stimulation of VEGF synthesis by insulin may lead to transient acceleration of retinal neovascularization in patients with diabetes after insulin therapy is instituted.

Tumor necrosis factor-alpha (TNF-α) is present at increased levels within the eye during the retinal processes of inflammation and angiogenesis. TNF-α promotes proliferation of trabecular meshwork cells and modulates the expression of trabecular meshwork, matrix metalloproteinases and tissue inhibitors. The integrin alpha 5 beta 3 (αVβ3) promotes angiogenesis in PDR as well as in AMD (Enaida, et al., Fukushima J Med Sci. 44 (1):43-52. (1998)).

There is therefore a need for therapeutics specific to cytokine, growth factors, and cell surface receptors that significantly reduce or eliminate deleterious side effects in the treatment of eye diseases and almost eliminate disease progression.

### BRIEF DESCRIPTION OF THE DRAWINGS

FIG. 1 is an illustration depicting the in vitro aptamer selection (SELEX™) process using pools of random sequence oligonucleotides;

FIG. 2 illustrates various strategies for synthesis of high molecular weight PEG-nucleic acid conjugates;

FIG. 3A is an illustration depicting the ARC82 TGFβ2 therapeutic aptamer (SEQ ID No. 151); and FIG. 3B is a graph depicting the plasma half-life profile of ARC82;

FIG. 4A is a flow chart illustrating S75 size exclusion chromatography; FIG. 4B is an elution profile of TGFβ2 in S75 size exclusion chromatography; FIG. 4C is an illustration depicting a polyacrylamide gel electrophoresis (PAGE) gel containing the TGFβ2 dimer PAGE band.

FIG. 5A is a graph depicting human (●) or rat (□) TGFβ-2 protein binding to increasing concentration of the ARC77 TGFβ-2 specific aptamer; FIG. 5B is a graph depicting competitive binding of non-radiolabeled ARC77 (∇) and ARC81 (▪)(fit to Equation 2 estimates of aptamer dissociation constants) competed with ³²P-labeled ARC77 for binding to human TGFβ2;

FIG. 6A is a graph depicting inhibition of TGFβ2 by the ARC77, ARC78 and ARC81 aptamers; FIG. 6B is a graph depicting inhibition of the antiproliferative effects of the human and rodent forms of TGFβ2 by the ARC77 aptamer; FIG. 6C is a graph depicting the dissociation constant (K_{d}) of ARC77 human wild type (WT), mouse (NTK) and N-terminal His tagged versions of human TGF2;

FIG. 7A is a graph depicting inhibition of the antiproliferative effects of low concentrations of rabbit aqueous humor by the ARC81 aptamer and the control, an anti-TGFβ2 antibody; FIGS. 7B and 7C are graphs illustrating dose-dependent rescue of 1.5% rabbit aqueous humor-mediated inhibition of MLEC proliferation by the ARC81 aptamer and the anti-TGFβ2 control antibody;

FIG. 8A is an illustration depicting minimization and mutagenesis/modification strategies for a modified TGFβ2 aptamer of SEQ ID NO:1; FIG. 8B is a table showing the dissociation constant (K_{d}) of the modified TGFβ2 aptamer of FIG. 8A for TGFβ1, TGFβ2 and TGFβ3; FIG. 8C is a graph depicting reversal of the inhibitory effect of TGFβ2 on MLEC cell proliferation by the modified TGFβ2 aptamer of FIG. 8A;

FIG. 9A is a graph depicting the stoichiometry of the TGFβ2 aptamer/TGFβ2 complex; FIG. 9B is a diagram illustrating the interaction between detectably labeled TGFβ2 aptamers and a TGF2 homodimer;

FIG. 10A is an illustration depicting the mapping of the aptamer binding site using the wild type, the N-terminal long tag and the N-terminal short tag variants human TGFβ2; FIG. 10B is a table listing the EC100 values, the dissociation constants and the IC₅₀ values for wild type TGFβ2, the N-terminal long tag variant of human TGFβ2, the N-terminal short tag variant of human TGFβ2, and two TGFβ2 mutants (K94N, S59T/R60K/K94N);

FIG. 11A is an illustration of a dot blot assay and a graph depicting TGF type III receptor blocking of the aptamer binding site; FIG. 11B is a diagram illustrating the potential overlap between the aptamer and TGF type III receptor binding sites;

FIG. 12 is an illustration of modified regions of the TGFβ2 aptamer ARC77 (SEQ ID No. 1);

FIG. 13A is a graph showing the binding curves of ARC 127 (SEQ ID NO:19-PEG-SEQ ID NO:35-PEG-SEQ ID NO:36) for the BB, AA and AB isoforms of human PDGF, and a Table listing the K_{d} values for ARC 127 (PEG-SEQ ID NO:19-PEG-SEQ ID NO:35-PEG-SEQ ID NO:36-3T) and the BB, AA and AB isoforms of PDGF; FIG. 13B is a graph showing the binding curves of ARC 127 (PEG-SEQ ID NO:19-PEG-SEQ ID NO:35-PEG-SEQ ID NO:36-3T)for the BB isoforms of human and rat PDGF, and a Table listing the K_{d} values for ARC 127 (PEG-SEQ ID NO:19-PEG-SEQ ID NO:35-PEG-SEQ ID NO:36-3T) and the human, rat and mouse BB isoforms of PDGF;

FIG. 14A is a graph depicting the ARC 127 (PEG-SEQ ID NO:19-PEG-SEQ ID NO:35-PEG-SEQ ID NO:36-3T)aptamer binding to human and rat PDGF; FIG. 14B is a graph comparing inhibition of PDGF-induced 3T3 cell proliferation by the ARC127 aptamer to inhibition of PDGF-induced 3T3 cell proliferation by a control antibody.

FIG. 15A is an image depicting migration of retinal pigmented epithelial (RPE) cells in the absence of PDGF; FIG. 15B is an image depicting migration of RPE cells in the presence of 100 ng/ml PDGF; FIG. 15C is an image depicting migration of RPE cells in the presence of PDGF and the ARC127 aptamer; FIG. 15D is an image depicting migration of RPE cells in the presence of PDGF and the ARC128 aptamer; FIGS. 15E and 15F are graphs depicting the effect of increasing PDGF concentrations on RPE cell migration.

FIG. 16 is a graph illustrating in vitro plasma stability of the all-DNA construct of the ARC127 aptamer (PEG-SEQ ID No. 19-PEG-SEQ ID NO:35-PEG-SEQ ID NO:36-3T) and the modified ARC127 aptamer;

FIG. 17 is a graph illustrating the concentration of ARC127 aptamer through 50 hours post dose via IV, IP and SC routes of administration;

FIG. 18 is a graph showing that ARC127 has measurable activity out to 48 hours in vivo;

FIGS. 19A and 19B are graphs showing the binding plots for the full-length TGFβ2 aptamer sequences shown in Table 5;

FIGS. 20A, 20B, and 20C are graphs showing the binding plots for the truncated TGFβ2 aptamers shown in Table 7;

FIGS. 21A, 21B, and 21C are graphs showing that the ARC117 and ARC119 aptamers have measurable activity out to 48 hours in vivo; and

FIGS. 22A, 22B, and 22C are graphs showing the in vivo activity of the ARC126 and ARC127 and NX1838 aptamers out to at least 25 days.

### SUMMARY OF THE INVENTION

The specificity of aptamers allows them to be used as therapeutics capable of binding specifically to cytokines, growth factors or cell surface proteins that promote scar tissue formation or other cellular events that lead to increased IOP in the glaucomatous eye. The present invention provides aptamer therapeutics with specific binding affinity to TGFβ2 and PDGF cytokines that contribute to post-surgical tissue scarring and can thus prevent increased IOP in the glaucomatous eye, and other pathologic processes of glaucoma.

In one embodiment, the present invention provides aptamer compositions capable of binding to TGFβ1, TGFβ2 or TFGβ3 useful in the treatment of diseases of the eye.

In one embodiment, the present invention provides aptamer compositions capable of binding to platelet derived growth factor (PDGF) useful in the treatment of diseases of the eye.

In one embodiment, the present invention provides aptamer compositions capable of binding to ICAM-1 useful in the treatment of diseases of the eye.

In one embodiment, the present invention provides aptamer compositions capable of binding to IGF-1 useful in the treatment of diseases of the eye.

In one embodiment, the present invention provides aptamer compositions capable of binding to VEGF/IVEGF-R useful in the treatment of diseases of the eye.

In one embodiment, the present invention provides aptamer compositions capable of binding to TNF-α useful in the treatment of diseases of the eye.

In one embodiment, the present invention provides aptamer compositions capable of binding to αVβ3 useful in the treatment of diseases of the eye.

In another embodiment, the present invention provides methods of treating subjects with the compositions of the present invention to treat proliferative disease involving TGFβ2-mediated cell proliferation.

In another embodiment, the present invention provides methods of treating subjects with the compositions of the present invention to treat proliferative disease involving PDGF -mediated cell proliferation.

In another embodiment, the present invention provides methods of treating subjects with the compositions of the present invention to treat proliferative disease involving ICAM-1-mediated cell proliferation.

In another embodiment, the present invention provides methods of treating subjects with the compositions of the present invention to treat proliferative disease involving IGF-1-mediated cell proliferation.

In another embodiment, the present invention provides methods of treating subjects with the compositions of the present invention to treat proliferative disease involving VEGF/IVEGF-R-mediated cell proliferation.

In another embodiment, the present invention provides methods of treating subjects with the compositions of the present invention to treat proliferative disease involving TNF-α-mediated cell proliferation.

In another embodiment, the present invention provides methods of treating subjects with the compositions of the present invention to treat proliferative disease involving αVβ3-mediated cell proliferation.

In another embodiment, the present invention provides nucleic acid therapeutic compositions and methods for delivering nucleic acid therapeutics capable of binding to cytokines, growth factors and cell surface receptors individually or combinations of two or more of PDGF, TGFβ1, TGFβ2, TGFβ3, ICAM-1, IGF1, VEGF-R, VEGF, TNFα, and αVβ₃, for the treatment of glaucoma and other proliferative diseases of the eye.

In another embodiment, the present invention provides nucleic acid therapeutic compositions and methods for delivering nucleic acid therapeutics capable of binding to PDGF and VEGF. In one embodiment, the nucleic acid therapeutic is a single nucleic acid aptamer that has one domain capable of binding to PDGF and a second domain capable of binding to VEGF. In another embodiment, the nucleic acid therapeutic is a solution that contains a first nucleic acid aptamer capable of binding PDGF and a second nucleic acid aptamer capable of binding VEGF, wherein the first and second nucleic acid aptamers are not the same nucleic acid aptamer.

In another embodiment, the present invention provides high molecular weight PEG-derivatized nucleic acid (e.g., aptamer) conjugates with improved pharmacological and pharmacodynamic properties and methods for producing such conjugates.

In one embodiment, the present invention provides high molecular weight PEG-nucleic acid (e.g., aptamer) conjugates and methods for producing such conjugates using a homo-bifunctional PEG to form a high molecular weight complex (i.e., a PEG-nucleic acid-PEG-nucleic acid-PEG-nucleic acid conjugate).

In one embodiment, the present invention provides high molecular weight PEG-nucleic acid (e.g., aptamer) conjugates and methods for producing such conjugates using a bi-reactive nucleic acid (i.e., a nucleic acid bearing two reactive sites) with a mono-functional PEG to form a multiple PEGylated conjugate (i.e., a PEG-nucleic acid-PEG conjugate).

In one embodiment, the high molecular weight PEG-nucleic acid conjugates of the present invention can be used as therapeutics in the prevention and/or treatment of ocular diseases and disorders.

In one aspect, high molecular weight PEG-aptamer compositions of the invention include a nucleic acid and a stabilizing moiety that is a linking moiety, wherein the linking moiety is not a nucleic acid molecule. In one embodiment, the linking moiety is polyalkylene glycol. Suitable polyalkylene glycols include, for example, polyethylene glycol (PEG). In some embodiments, the polyethylene glycol (PEG) linking moiety is multi-activated. For example, the PEG linking moiety is bi-activated. In one embodiment, the first and second portions of an aptamer are linked by a PEG linking moiety, such that the primary structure of the aptamer composition is a linear arrangement in which the first moiety is linked to a first terminus of the PEG linking moiety and the second moiety is linked to a second terminus of the PEG linking moiety. In some aspects, there is more than one PEG moiety separating more than two nucleic acid aptamer moieties, for example, the linear arrangement of the high molecular weight aptamer composition is: nucleic acid-PEG-nucleic acid-PEG-nucleic acid. In some embodiments, the linear arrangement of the high molecular weight aptamer composition is: PEG-nucleic acid-PEG-nucleic acid-PEG-nucleic acid. In some embodiments, the high molecular weight aptamer composition has a molecular weight selected from the group consisting of greater than 10 kD, greater than 20 kD, greater than 40 kD and greater than 80 kD. Some high molecular weight aptamer compositions according to this aspect of the invention are capable of binding to platelet derived growth factor (PDGF). Some high molecular weight aptamer compositions according to this aspect of the invention are capable of binding to TGFβ2.

In another aspect, the invention provides high molecular weight PEG-aptamer compositions that include an aptamer, and two or more non-nucleic acid stabilizing moieties. Suitable stabilizing moieties include, for example, a polyalkylene glycol. In one embodiment, the stabilizing moiety is polyethylene glycol (PEG). In one embodiment, the aptamer is multi-activated. For example, the aptamer is bi-activated.

The present invention also provides therapeutic compositions that include the high molecular weight PEG-aptamer compositions described herein.

In another aspect, the present invention provides methods of treating disease in a subject comprising the steps of administering a therapeutically effective amount of a high molecular weight PEG-aptamer compositions described herein.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

All publications, patent applications, patents, and other references mentioned herein are incorporated by reference in their entirety. Citation of publications and patent documents is not intended as an admission that any is pertinent prior art, nor does it constitute any admission as to the contents or date of the same. In the case of conflict, the present Specification, including definitions, will control. In addition, the materials, methods, and examples described below are illustrative only and not intended to be limiting.

The SELEX™ Process

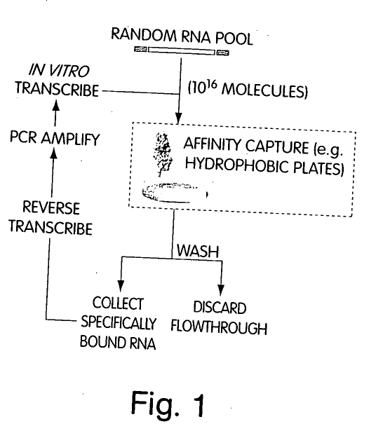

A suitable method for generating an aptamer is with the process entitled “Systematic Evolution of Ligands by EXponential Enrichment” (“SELEX™”) generally depicted in FIG. 1. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotide templates comprising randomized sequences derived from chemical synthesis on a standard DNA synthesizer. In some examples, a population of 100% random oligonucleotides is screened. In others, each oligonucleotide in the population comprises a random sequence and at least one fixed sequence at its 5′ and/or 3′ end which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores, sites for selective binding to affinity columns, and other sequences to facilitate cloning and/or sequencing of an oligonucleotide of interest.

The random sequence portion of the oligonucleotide can be of any length and can comprise ribonucleotides and/or deoxyribonucleotides and can include modified or non-natural nucleotides or nucleotide analogs. See, e.g., U.S. Pat. Nos. 5,958,691; 5,660,985; 5,958,691; 5,698,687; 5,817,635; and 5,672,695, PCT publication WO 92/07065. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

Template molecules typically contain fixed 5′ and 3′ terminal sequences which flank an internal region of 30-50 random nucleotides. A standard (1 μmole) scale synthesis will yield 10¹⁵-10¹⁶ individual template molecules, sufficient for most SELEX experiments. The RNA library is generated from this starting library by in vitro transcription using recombinant T7 RNA polymerase. This library is then mixed with the target under conditions favorable for binding and subjected to step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

Within a nucleic acid mixture containing a large number of possible sequences and structures, there is a wide range of binding affinities for a given target. A nucleic acid mixture comprising, for example a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20-50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. For example, see U.S. Pat. No. 5,580,737 which discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, which describes oligonucleotides containing nucleotide derivatives chemically modified at the 2′ position of ribose, 5′ position of pyrimidines and 8′ positions of purines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping. In preferred embodiments of the instant invention, the nucleic acid ligands are RNA molecules that are 2′-fluoro (2′-F) modified on the sugar moiety of pyrimidine residues.

The modifications can be pre- or post-SELEX process modifications. Pre-SELEX process modifications yield nucleic acid ligands with both specificity for their SELEX target and improved in vivo stability. Post-SELEX process modifications made to 2′-OH nucleic acid ligands can result in improved in vivo stability without adversely affecting the binding capacity of the nucleic acid ligand.

Other modifications are known to one of ordinary skill in the art. Such modifications may be made post-SELEX process (modification of previously identified unmodified ligands) or by incorporation into the SELEX process.

The SELEX method encompasses combining selected oligonucleotides with other selected oligonucleotides and non-oligonucleotide functional units as described in U.S. Pat. No. 5,637,459 and U.S. Pat. No. 5,683,867. The SELEX method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications allow the combination of a broad array of shapes and other properties, and the efficient amplification and replication properties, of oligonucleotides with the desirable properties of other molecules.

The identification of nucleic acid ligands to small, flexible peptides via the SELEX method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute intemucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2-fluoro-ribonucleotide oligomer molecules can increase the sensitivity of a nucleic acid sensor molecule for a target molecule by ten- to- one hundred-fold over those generated using unsubstituted ribo- or deoxyribo-oligonucteotides (Pagratis, et al., Nat. Biotechnol. 15:68-73 (1997)), providing additional binding interactions with a target molecule and increasing the stability of the secondary structure(s) of the nucleic acid sensor molecule (Kraus, et al., Journal of Immunology 160:5209-5212 (1998); Pieken, et al, Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol 15:68-73 (1997)).

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase or modified T7 RNA polymerases and purified. In one example, the 5′ fixed:random:3′-fixed sequence is separated by random sequence having 30 to 50 nucleotides.

2′O-Me SELEX™

In addition, the SELEX™ method can be performed to generate 2′ modified aptamers as described in U.S. Ser. No. 60/430,761, filed Dec. 3, 2002, U.S. Provisional Patent Application Ser. No. 60/487,474, filed Jul. 15, 2003, and U.S. Provisional Patent Application Ser. No. 60/517,039, filed Nov. 4, 2003, and U.S. patent application Ser. No. 10/729,581, filed Dec. 3, 2003, each of which is herein incorporated by reference in its entirety.

The present invention also provides materials and methods to produce stabilized oligonucleotides, including, e.g., aptamers, that contain modified nucleotides (e.g., nucleotides which have a modification at the 2′position) which make the oligonucleotide more stable than the unmodified oligonucleotide. The stabilized oligonucleotides produced by the materials and methods of the present invention are also more stable to enzymatic and chemical degradation as well as thermal and physical degradation. For example, oligonucleotides containing 2′-O-methyl nucleotides are nuclease-resistant and inexpensive to synthesize. Although 2′-O-methyl nucleotides are ubiquitous in biological systems, natural polymerases do not accept 2′-O-methyl NTPs as substrates under physiological conditions, thus there are no safety concerns over the recycling of 2′-O-methyl nucleotides into host DNA.

In one embodiment, the present invention provides combinations of 2′-OH, 2′-F, 2′-deoxy, and 2′-OMe modifications of the ATP, GTP, CTP, TTP, and UTP nucleotides. In another embodiment, the present invention provides combinations of 2′-OH, 2′-F, 2′-deoxy, 2′-OMe, 2′-NH₂, and 2′-methoxyethyl modifications of the ATP, GTP, CTP, TTP, and UTP nucleotides. In one embodiment, the present invention provides 5⁶ combinations of 2′-OH, 2′-F, 2′-deoxy, 2′-OMe, 2′-NH₂, and 2′-methoxyethyl modifications the ATP, GTP, CTP, TTP, and UTP nucleotides.

2′ modified aptamers of the invention are created using modified polymerases, such as, e.g., a modified T7 polymerase, having a higher incorporation rate of modified nucleotides having bulky substituents at the furanose 2′ position, than wild-type polymerases. For example, a double T7 polymerase mutant (Y639F/H784A) having the histidine at position 784 changed to an alanine, or other small amino acid, residue, in addition to the Y639F mutation has been described for incorporation of bulky 2′ substituents and has been used to incorporate modified pyrimidine NTPs. A single mutant T7 polymerase (H784A) having the histidine at position 784 changed to an alanine residue has also been described. (Padilla et al., Nucleic Acids Research, 2002, 30: 138). In both the Y639F/H784A double mutant and H784A single mutant T7 polymerases, the change to smaller amino acid residues allows for the incorporation of bulkier nucleotide substrates, e.g., 2′-O methyl substituted nucleotides.

Another important factor in the production of 2′-modified aptamers is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

Aptamers with Binding Affinity to TGFβ2 and PDGF

The present invention provides modified and unmodified nucleic acid aptamer therapeutics capable of binding to human cytokines, growth factors or cell surface proteins implicated in diseases of the eye. In one embodiment, the aptamers of the invention are capable of binding to TGFβ2 with high affinity and reversing TGFβ2-mediated inhibition of mink lung epithelial cells (MLEC) proliferation in vitro. These aptamers can be generated using a process termed “Systematic Evolution of Ligands by Exponential Enrichment” (the SELEX™ process) depicted in FIG. 1.

The modified RNA aptamers of the present invention bind native human TGFβ2. For bio-chemical characterization of these aptamers, two forms of mature TGFβ2 were generated, native and N-terminal his-tagged versions, in E. coli. After refolding and purification, functional TGFβ2s were obtained. These TGFβ2 proteins were active in cell based assay. N-terminal tags affected both activity and aptamer binding while the affinity to aptamer was decreased to a much larger extent. Further two mutant TGFβ2s (K94N, S59T/R60K/K94N) were generated based on known isoforms of TGFβ2. The K94N mutant was capable of binding to the aptamers with comparable affinity with that of native TGFβ2, whereas the S59T/R60K/K94N mutant had significantly reduced affinity to the aptamers. Similarly, the aptamers blocked the bioactivity of native and K94N TGFβ2s with higher potencies than that of S59T/R60K/K94N mutant in a cell based assay. Based on published crystal structure, two substitutions at positions 59 and 60 reside near the dimer interface and adjacent to the N-terminus of TGFβ2 and the other substitution at position 94 is near the type II receptor binding site. Binding competition assay with soluble TGF-β receptors revealed that type III receptor competes with the aptamer binding, but not type II receptor. We also demonstrated that two aptamers bind to one species of dimer of TGFβ2 but not to the other two species of dimer. The aptamers of the present invention are believed to bind TGFβ2 near or at the TGFβ type III receptor binding site and block its biological function.

The aptamers with specificity and binding affinity to TGFβ2 of the present invention are selected by the SELEX process described above. As part of the SELEX process the sequences selected to bind to TGFβ2 are then minimized to determine the minimal sequence having binding affinity, and optimized by performing random or directed mutagenesis of the minimized sequence to determine if increases of affinity or alternatively which positions in the sequence are essential for binding activity. Additionally, selections can be performed with sequences incorporating modified sequences to stabilize the aptamer molecules against degradation in vivo.

The selected aptamers having the highest affinity and specific binding as demonstrated by biological assays as described in the examples below are suitable therapeutics for treating conditions in which TGFβ2 is involved in pathogenesis. Alternatively, the aptamers selected for specificity to PDGF are suitable therapeutics for treating conditions in which PDGF is involved in pathogenesis.

Some aptamer compositions of the present invention have binding affinity and specificity to certain dimers of platelet derived growth factor (PDGF). The aptamer compositions of the present invention have binding affinity to the PDGF BB homodimer and to the AB heterodimer but not to the AA homodimer.

The aptamer compositions of the present invention can be used as therapeutic compositions to treat subjects with ocular disease involving TGFβ2- or PDGF-mediated proliferative disease. For example, aptamers that are selective for PDGF can be used in the treatment of eye diseases such as PVR, PDR and AMD. In addition, PDGF aptamers can be administered alone, or in conjunction with other known therapies, such as anti-VEGF therapies, anti-inflammatory agents, anti-proliferative agents, antibacterial agents, antifingal agents and antimicrobial agents. TGFβ2 aptamers can be used to treat damage or injury that occurs after a trabeculectomy, such as, for example, scarring. Thus, TGFβ2 aptamers can be administered prior to, during, or after a trabeculectomy. The TGFβ2 aptamers can be administered alone, or in combination with other known therapies, such as, for example, anti-inflammatory agents, anti-proliferative agents, antibacterial agents, antifugal agents and antimicrobial agents.

TGFβ2-Specific Aptamer Therapeutics in Trabeculectomy

TGFβ2 aptamers of the invention may be applied in eye drop form. This method is non-invasive and will increase ease of patient compliance. If administered via microdevice, microparticle or sponge, application can occur during surgery as described above.

The TGFβ2 aptamer may be administered lyophilized in polymer sustained delivery devices with delivery solution which will mix before being released into the eye. Sustained delivery from polymer matrixes offers the advantage of targeting specific tissues and increasing the comfort and compliance of patients. PLGA (polylacticcoglycolic acid) is the encapsulation matrix of choice as it is FDA approved and used as a suture material since the 1970s and as a scaffold in tissue engineering. It is biocompatible and well studied for its toxicology and degradation kinetics.

In addition, the TGFβ2 aptamer may be administered lyophilized in polymer contact lens sustained delivery device. The contact lens aptamer therapeutic delivery device will increase patient dosing compliance and help health care providers with ease of application of the aptamer therapeutic while assuring a constant zero-order delivery of the aptamer therapeutic.

Subconjunctival administration may be used in a volume of 100 μl in the manner described above for 5-FU.

The TGFβ2 aptamer is administered at a determined effective dose as eye drops, with a microdevice, microparticle or sponge or subconjunctivally near the anterior segment. It may be lyophilized for storage and reconstituted and administered in a sterile, aqueous, preservative free bicarbonate-buffered solution. Dosing for the TGFβ2 aptamer is in the range of 0.1-200 mg kg⁻¹. Preferred dosing for animals of the TGFβ2 aptamer is in the range of 0.1-100 mg kg⁻¹. More preferred dosing for animals of the TGFβ2 aptamer is in the range of 0.1-10 mg kg⁻¹. Most preferred dosing for animals of the TGFβ2 aptamer is in the range of 0.1-1 mg kg⁻¹. Dosing for humans is in the range of 7-70 mg kg⁻¹.

PDGF Aptamer Therapeutics in Age-Related Macular Degeneration (AMD).

PDGF plays an important role in the pathogenesis of AMD in synergistic action with other growth factors such as VEGF. Hypoxia increases expression of PDGF and PDGF directly acts on endothelial cells through PDGFR-b to induce angiogenesis. PDGF acts on fibroblasts, smooth muscle cells, neuroglial cells, and stimulates proliferation of connective-tissue cells.

Dosing of the PDGF aptamer for animals is in the range of 0.1-200 mg kg⁻¹. Preferred dosing of the PDGF aptamer for animals is in the range of 0.1-100 mg kg⁻¹. More preferred dosing of the PDGF aptamer for animals is in the range of 0.1-10 mg kg⁻¹. Most preferred dosing of the PDGF aptamer for animals is in the range of 0.1-11 mg kg⁻¹. Human dosing is in the range of 7-70 mg kg⁻¹.

The PDGF aptamer can be injected intravitreally in a single dose at a determined effective concentration in 100ul delivery volume. Injection is through the pars plana using a 30-gauge needle and tuberculin syringe after instilling topical anesthesia and 5% povidone iodine solution. Before dosing, the vial stopper is wiped with 70% alcohol. PDGF aptamer can be stored lyophilized and dissolved into a ready to use sterile solution composed of 10 mM sodium phosphate and 0.9% sodium chloride buffer. Intravitreal administration is used in many intraocular diseases using allowing efficient penetration into the eye. The tight complexes of the retinal pigment epithelium and retinal capillaries serve as the blood-ocular barrier, which inhibits penetration of therapeutic agents into the vitreous. This route of administration avoids the potential side effects that may be experienced with systemic administration and allows for efficient targeting of therapeutic area.

Additionally, the PDGF aptamer may be delivered transsclerally. Transscleral delivery is a viable mode of administering therapeutics to the posterior segment. The sclera has a large and accessible surface area and a high degree of hydration that renders it conducive to water-soluble substances. It is also relatively devoid of cells and thus has few proteolytic enzymes or protein-binding sites that can bind or sequester therapeutic agents. Scleral permeability permits both small and large molecular weight agents and permeability does not decline with age which is favorable for the treatment of chronic diseases such as AMD which occur increasingly in the elderly. It is non-destructive, minimally invasive and achieves targeted delivery. Additionally, slow release transscleral devices allow for consistent release of therapeutic PDGF aptamer.

PDGF Aptamer Therapeutics in Proliferative Vitreoretinopathy (PVR).

Surgery for PVR begins with a vitrectomy (pars plana vitrectomy) procedure, following the vitrectomy procedure, the surgeon usually instills special gases or fluids into the eye to help flatten the retina and keep it reattached to the outer wall of the eye. The PDGF aptamer can be injected intravitreally in either before or after vitrectomy or both in a single dose at a determined effective concentration in 100ul delivery volume. Injection is through the pars plana using a 30-gauge needle and tuberculin syringe after instilling topical anesthesia and 5% povidone iodine solution. Before dosing, the vial stopper is wiped with 70% alcohol. PDGF aptamer can be stored lyophilized and dissolved into a ready to use sterile solution composed of 10 mM sodium phosphate and 0.9% sodium chloride buffer. Intravitreal administration is used in many intraocular diseases using allowing efficient penetration into the eye. The tight complexes of the retinal pigment epithelium and retinal capillaries serve as the blood-ocular barrier, which inhibits penetration of therapeutic agents into the vitreous. This route of administration avoids the potential side effects that may be experienced with systemic administration and allows for efficient targeting of therapeutic area.

PDGF aptamer may be delivered via a biodegradable microsize polymer system. The aptamer may be encapsulated in polymer with a predetermined rate of release. This ensures localized delivery, consistent dosing and assured compliance. Delivery can occur via a polymer coated pellet with variable permeability to the aptamer. This implant can be surgically inserted through the pars plana during vitrectomy.

Additionally, the PDGF aptamer may be delivered transsclerally. Transscleral delivery is a viable mode of administering therapeutics to the posterior segment. The sclera has a large and accessible surface area and a high degree of hydration that renders it conducive to water-soluble substances. It is also relatively devoid of cells and thus has few proteolytic enzymes or protein-binding sites that can bind or sequester therapeutic agents. Scleral permeability permits both small and large molecular weight agents and permeability does not decline with age which is favorable for the treatment of chronic diseases such as AMD which occur increasingly in the elderly. It is non-destructive, minimally invasive and achieves targeted delivery. Additionally, slow release transscleral devices allow for consistent release of therapeutic PDGF aptamer.

PDGF Aptamer Therapeutics in Proliferative Diabetic Retinopathy (PDR).

The PDGF aptamer may be injected intravitreally in a single dose at a determined effective concentration in 100ul delivery volume. Injection is through the pars plana using a 30-gauge needle and tuberculin syringe after instilling topical anesthesia and 5% povidone iodine solution. Before dosing, the vial stopper is wiped with 70% alcohol. PDGF aptamer can be stored lyophilized and dissolved into a ready to use sterile solution composed of 10 mM sodium phosphate and 0.9% sodium chloride buffer. Intravitreal administration is used in many intraocular diseases using allowing efficient penetration into the eye. The tight complexes of the retinal pigment epithelium and retinal capillaries serve as the blood-ocular barrier, which inhibits penetration of therapeutic agents into the vitreous. This route of administration avoids the potential side effects that may be experienced with systemic administration and allows for efficient targeting of therapeutic area.

Additionally, the PDGF aptamer may be delivered transsclerally. Transscleral delivery is a viable mode of administering therapeutics to the posterior segment. The sclera has a large and accessible surface area and a high degree of hydration that renders it conducive to water-soluble substances. It is also relatively devoid of cells and thus has few proteolytic enzymes or protein-binding sites that can bind or sequester therapeutic agents. Scleral permeability permits both small and large molecular weight agents and permeability does not decline with age which is favorable for the treatment of chronic diseases such as AMD which occur increasingly in the elderly. It is non-destructive, minimally invasive and achieves targeted delivery. Additionally, slow release transscleral devices allow for consistent release of therapeutic PDGF aptamer.

Aptamers with Binding Specificity to Cytokines Growth Factors and Cell Surface Proteins

Cytokines, cell surface proteins and growth factors implicated in various ocular diseases include ICAM-1, TGFβ1, TGFβ2, TGFβ3, IGF-1, VEGF/NVEGF-R, TNF-α, angiopoietin and αVβ3. Intercellular Adhesion Molecule 1 (ICAM-1) is a 76 to 115 kDa surface glycoprotein with five extracellular immunoglobulin-like domains plays a particularly important role in diabetic retinopathy. ICAM-1-mediated leukostasis is causative in the pathogenesis of diabetic retinopathy. ICAM-1 interactions with β2 integrins located on the surface of leukocytes (neutrophils, basophils, lymphocytes, eosinophils, monocytes) are important for their firm adhesion to the endothelium and their transendothelial migration to sites of inflammation. ICAM-1 facilitates adhesion of leukocytes to the retinal vasculature in diabetic retinopathy and is involved with retinal endothelial cell injury and death via lesions that produce irreversible retinal ischemia through inability of capillaries to support blood flow. Inhibition of ICAM-1 bioactivity blocks diabetic retinal leukostasis and potently prevents blood-retinal barrier breakdown.

Insulin-like Growth Factor-1 (IGF-1) is a 7.5 kDa peptide, having 50% homology to proinsulin (50%) and is produced primarily in the liver under control of growth hormone. IGF-1 is a potent mitogen/stimulator of cell proliferation and a strong anti-apoptotic agent. Its function is modulated by six IGF binding proteins (IGFBPs) and its levels are influenced by developmental stage and nutrition. Its effects range from cell growth and protection, resistance to oxidative stress, promoting growth of bone and muscle, and protecting neuronal cells. IGF-1 is implicated in angiogenesis with VEGF playing a role in proliferative diabetic retinopathy (PDR). PDR is a complication of diabetes that is caused by changes in the blood vessels in the retina. When blood vessels in the retina are damaged, they may leak blood and grow fragile, brush-like branches and scar tissue. This can blur or distort vision images that the retina sends to the brain.

Vascular endothelial growth factor and its receptor (VEGF and VEGF/R) transcription are enhanced by advanced glycation end products and by insulin. The accumulation of advanced glycation end products in the diabetic retina contributes to neovascularization, which can result in loss of vision. The stimulation of VEGF synthesis by insulin may lead to transient acceleration of retinal neovascularization in patients with diabetes after insulin therapy is instituted. An aptamer directed to VEGF-165 was found to have a dissociation constant (K_{d}) of 300 pM and an IC50 value of 1 nM.

Tumor necrosis factor-alpha (TNF-α) is present at increased levels within the eye during retinal processes of inflammation and angiogenesis. TNF-α promotes proliferation of trabecular meshwork cells; modulates trabecular meshwork, matrix metalloproteinases and tissue inhibitor expression; increases MMP-1, 3, and 9 and TIMP-1 expression, decreases TIMP-2. Angiopoietin is an angiogenic growth factor that occurs in two forms, Ang-1 and Ang-2. An aptamer directed to Angiopoietin was found to have a dissociation constant (K_{d}) of 10 nM and the ability to block Ang1- and Ang-2 mediated inhibition of apoptosis in TNF-α treated HUVEC cells. The integrin alpha 5 beta 3 (αVβ3) promotes angiogenesis in PDR as well as in AMD (Enaida, et al., Fukushima J Med Sci. 44 (1):43-52. (1998)).

Aptamers of the present invention are capable of binding to ICAM-1, TGFβ1, TGFβ2, TGFβ3, IGF-1, VEGF/VEGF-R, TNF-α, and αVβ3, individually or in combination to one or more, and inhibit their signaling activities and therefore their role in ocular disease pathogenesis.

PEG-Derivatized Nucleic Acids

Derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamer compositions of the invention may be derivatized with polyalkylene glycol (PAG) moieties. Examples of PAG-derivatized nucleic acids are found in U.S. patent application Ser. No. 10/718,833, filed on Nov. 21, 2003, which is herein incorporated by reference in its entirety. Typical polymers used in the invention include poly(ethylene glycol) (PEG), also known as or poly(ethylene oxide) (PEO) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpoly(ethylene glycol), can also be represented as HO-PEG-OH, where it is understood that the -PEG- symbol represents the following structural unit: CH₂CH₂O—(CH₂CH₂O)ₙ—CH₂CH₂—where n typically ranges f rom about 4 to about 10,000.

As shown, the PEG molecule is di-functional and is sometimes referred to as “PEG diol.” The terminal portions of the PEG molecule are relatively non-reactive hydroxyl moieties, the —OH groups, that can be activated, or converted to functional moieties, for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively nonreactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics which generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with non-reactive methoxy end moiety, —OCH₃. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J Org. Chem., 60:331-336 (1995). PAG conjugates are often used not only to enhance solubility and stability but also to prolong the blood circulation half-life of molecules.

Polyalkylated compounds of the invention are typically between 5 and 80 kD in size. Other PAG compounds of the invention are between 10 and 80 kD in size. Still other PAG compounds of the invention are between 10 and 60 kD in size. For example, a PAG polymer may be at least 10, 20, 30, 40, 50, 60, or 80 kD in size. Such polymers can be linear or branched.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa (further increases have minimal beneficial effect as half life becomes defined by macrophage-mediated metabolism rather than elimination via the kidneys).

Production of high molecular weight PEGs (>10 kDa) can be difficult, inefficient, and expensive. As a route towards the synthesis of high molecular weight PEG-nucleic acid conjugates, previous work has been focused towards the generation of higher molecular weight activated PEGs. One method for generating such molecules involves the formation of a branched activated PEG in which two or more PEGs are attached to a central core carrying the activated group. The terminal portions of these higher molecular weight PEG molecules, i e., the relatively non-reactive hydroxyl (—OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol.2:214-221, 2003).

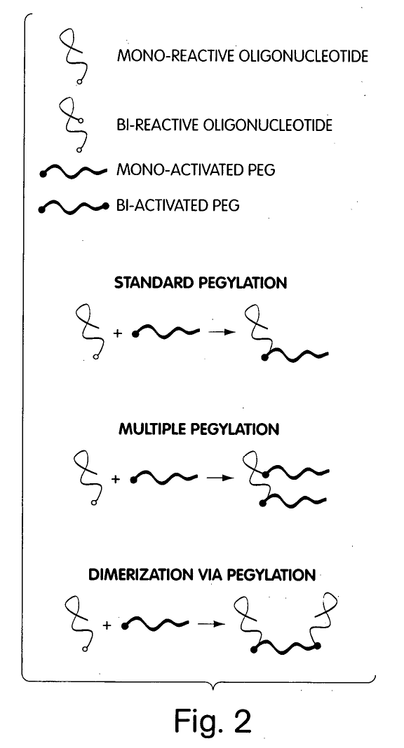

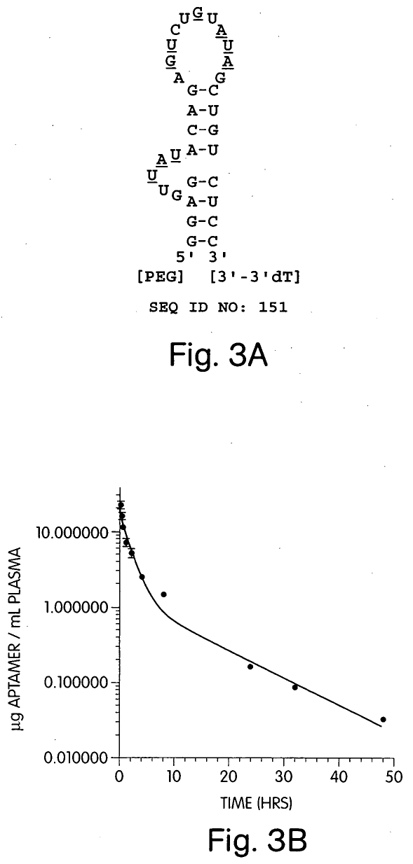

The present invention provides another cost effective route to the synthesis of high molecular weight PEG-nucleic acid (preferably, aptamer) conjugates including multiply PEGylated nucleic acids (as illustrated, e.g., in FIG. 2). The present invention also encompasses PEG-linked multimeric oligonucleotides, e.g., dimerized aptamers (as also illustrated, e.g., in FIG. 2). The present invention also relates to high molecular weight compositions where a PEG stabilizing moiety is a linker which separates different portions of an aptamer, e.g., the PEG is conjugated within a single aptamer sequence, such that the linear arrangement of the high molecular weight aptamer composition is, e.g., nucleic acid-PEG-nucleic acid-PEG-nucleic acid.

High molecular weight compositions of the invention include those having a molecular weight of at least 10 kD. Compositions typically have a molecular weight between 10 and 80 kD in size. High molecular weight compositions of the invention are at least 10, 20, 30, 40, 50, 60, or 80 kD in size.

A stabilizing moiety is a molecule, or portion of a molecule, which improves pharmacokinetic and pharmacodynamic properties of the high molecular weight aptamer compositions of the invention. In some cases, a stabilizing moiety is a molecule or portion of a molecule which brings two or more aptamers, or aptamer domains, into proximity, or provides decreased overall rotational freedom of the high molecular weight aptamer compositions of the invention. A stabilizing moiety can be a polyalkylene glycol, such a polyethylene glycol, which can be linear or branched, a homopolymer or a heteropolymer. Other stabilizing moieties include polymers such as peptide nucleic acids (PNA). Oligonucleotides can also be stabilizing moieties; such oligonucleotides can include modified nucleotides, and/or modified linkages, such as phosphothioates. A stabilizing moiety can be an integral part of an aptamer composition, i.e., it is covalently bonded to the aptamer.

Compositions of the invention include high molecular weight aptamer compositions in which two or more nucleic acid moieties are covalently conjugated to at least one polyalkylene glycol moiety. The polyalkylene glycol moieties serve as stabilizing moieties. In compositions where a polyalkylene glycol moiety is covalently bound at either end to an aptamer, such that the polyalkylene glycol joins the nucleic acid moieties together in one molecule, the polyalkylene glycol is said to be a linking moiety. In such compositions, the primary structure of the covalent molecule includes the linear arrangement nucleic acid-PAG-nucleic acid. One example is a composition having the primary structure nucleic acid-PEG-nucleic acid. Another example is a linear arrangement of: nucleic acid-PEG-nucleic acid-PEG-nucleic acid.

To produce the nucleic acid-PEG-nucleic acid conjugate, the nucleic acid is originally synthesized such that it bears a single reactive site (e.g., it is mono-activated). In a preferred embodiment, this reactive site is an amino group introduced at the 5′-terminus by addition of a modifier phosphoramidite as the last step in solid phase synthesis of the oligonucleotide. Following deprotection and purification of the modified oligonucleotide, it is reconstituted at high concentration in a solution that minimizes spontaneous hydrolysis of the activated PEG. In a preferred embodiment, the concentration of oligonucleotide is 1 mM and the reconstituted solution contains 200 mM NaHCO₃-buffer, pH 8.3. Synthesis of the conjugate is initiated by slow, step-wise addition of highly purified bi-functional PEG. In a preferred embodiment, the PEG diol is activated at both ends (bi-activated) by derivatization with succinimidyl propionate. Following reaction, the PEG-nucleic acid conjugate is purified by gel electrophoresis or liquid chromatography to separate fully-, partially-, and un-conjugated species. Multiple PAG molecules concatenated (e.g., as random or block copolymers) or smaller PAG chains can be linked to achieve various lengths (or molecular weights). Non-PAG linkers can be used between PAG chains of varying lengths.

One high molecular weight composition of the invention has the following structure:

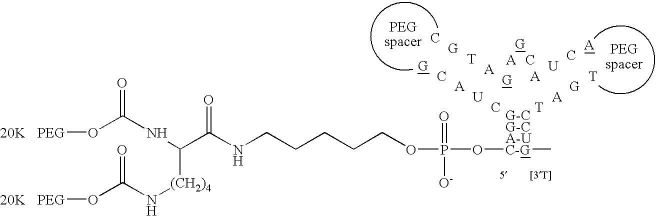

(40 K branched PEG-SEQ ID NO:19-0.1 kDa PEG-SEQ ID NO:35-0.1 kDa PEG-SEQ ID NO:36). 2′-O-methyl modified nucleotides are underlined and 2′-fluoro modified nucleotides are italicized. The 2′-O-methyl, 2′-fluoro modifications stabilize the aptamer against nucleases and increase its half life in vivo. The 3′-3′-dT cap also increases exonuclease resistance. See, e.g., U.S. Pat. Nos. 5,674,685; 5,668,264; 6,207,816; and 6,229,002, each of which is incorporated by reference herein in its entirety.

PAG-derivatization of a reactive nucleic acid. High molecular weight PAG-nucleic acid-PAG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation as illustrated in FIG. 2. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully-, partially-, and un-conjugated species. FIG. 2 illustrates two strategies for synthesizing PEGylated nucleic acid aptamers.

The linking domains can also have one ore more polyalkylene glycol moieties attached thereto. Such PAGs can be of varying lengths and may be used in appropriate combinations to achieve the desired molecular weight of the composition.

The effect of a particular linker can be influenced by both its chemical composition and length. A linker that is too long, too short, or forms unfavorable steric and/or ionic interactions with the target will preclude the formation of complex between aptamer and target. A linker, which is longer than necessary to span the distance between nucleic acids may reduce binding stability by diminishing the effective concentration of the ligand. Thus, it is often necessary to optimize linker compositions and lengths in order to maximize the affinity of a to target.

Pharmaceutical Compositions

The invention also includes pharmaceutical compositions containing aptamer molecules. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. Compositions of the invention are useful for administration to a subject suffering from, or predisposed to, a disease or disorder which is related to or derived from a target to which the aptamers specifically bind.

For example, the target is a protein involved with a pathology, for example, the target protein causes the pathology.

Compositions of the invention can be used in a method for treating a patient having a pathology. The method involves administering to the patient a composition comprising aptamers that bind a target (e.g., a protein) involved with the pathology, so that binding of the composition to the target alters the biological function of the target, thereby treating the pathology.

The patient having a pathology, e.g. the patient treated by the methods of this invention can be a mammal, or more particularly, a human.

In practice, the compounds or their pharmaceutically acceptable salts, are administered in amounts which will be sufficient to inhibit growth factor activity, for example TGFβ2-, mediated cell proliferation in glaucoma and other proliferative diseases of the eye.

One aspect of the invention comprises an aptamer composition of the invention in combination with other treatments for ocular diseases. The aptamer composition of the invention may contain, for example, more than one aptamer. In some examples, an aptamer composition of the invention, containing one or more compounds of the invention, is administered in combination with surgery, or with another useful composition such as an anti-inflammatory agent, an immunosuppressant, an antiviral agent, or the like. Furthermore, the compounds of the invention may be administered in combination with a chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

“Combination therapy” (or “co-therapy”) includes the administration of an aptamer composition of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical. “Combination therapy” also can embrace the administration of the therapeutic agents as described above in further combination with other biologically active ingredients and non-drug therapies (e.g., surgery). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The compounds of the invention and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

Preferably, ocular therapeutics are administered topically or by subconjunctival injection. Repeated topical applications of most ocular drugs result in intraocular drug levels comparable to those achieved with subconjunctival injections, but subconjunctival injections offer an advantage in the administration of drugs with poor intraocular penetration (e.g., antibiotics). By subconjunctival injection, high local concentrations of drug can be obtained with the use of small quantities of medication, so that adverse systemic effects are avoided. High tissue concentrations can also be obtained with drugs that poorly penetrate the epithelial layer of the cornea or conjunctiva. This method is useful in patients who do not reliably use topical medication. Intraocular drugs can be injected at the conclusion of surgery to avoid the necessity of topical or systemic drug therapy. Subconjunctival injection involves passing the needle between the anterior conjunctiva and Tenon's capsule. This can be performed through the lid or directly into the subconjunctival space. Tenon's capsule lies between the injected drug and the globe of the eye, so the amount of drug absorbed across the sclera is minimized. In fact, the mechanism of drug absorption after subconjunctival injection may be simple leakage of drug through the needle puncture site with subsequent absorption through the cornea.

A variety of ocular diseases are treated with subconjunctival corticosteroids. For corticosteroids, a subconjunctivally administered drug does penetrate the underlying sclera, which suggests a rationale for placing the drug directly adjacent to the site of inflammation rather than injecting it randomly. Subconjunctival injection of 5-flourouracil, an antifibroblast agent, is sometimes used after high-risk trabeculectomy surgeries for glaucoma. Subconjunctival anesthesia is now used as an alternative to peribulbar or retrobulbar anesthesia for trabeculectomy or cataract surgery.

Subconjunctival drug administration is useful in the treatment of severe corneal disease, such as bacterial ulcers. Much higher concentrations of antibiotics can be achieved in the affected corneal tissues with subconjunctival injection than can be obtained by systemic drug administration. Subconjunctival antibiotic administration is also useful as an initial supplement to the systemic or intravitreal antibiotic treatment of bacterial endophthalmitis.

The compositions and combination therapies of the invention may be administered in combination with a variety of pharmaceutical excipients, including stabilizing agents, carriers and/or encapsulation formulations as described herein.

The pharmaceutical forms suitable for injectable use must be sterile and must be fluid to the extent that easy syringability exists. It must be stable under the conditions of manufacture and storage and must be preserved against the contaminating action of microorganisms, such as bacteria and fungi.

Therapeutic or pharmacological compositions of the present invention will generally comprise an effective amount of the component(s) of the combination therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, cremes, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or sponge.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer-toxin and/or riboreporter molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross- linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1,000.0 mg of active ingredient. Effective plasma levels of the compounds of the present invention range from 0.002 mg to 50 mg per kg of body weight per day. Compounds of the present invention may be administered in a single daily dose, or the total daily dosage may be administered in divided doses of two, three or four times daily.

### EXAMPLES

### Example 1

### Purified and Characterized TGFβ2 Protein

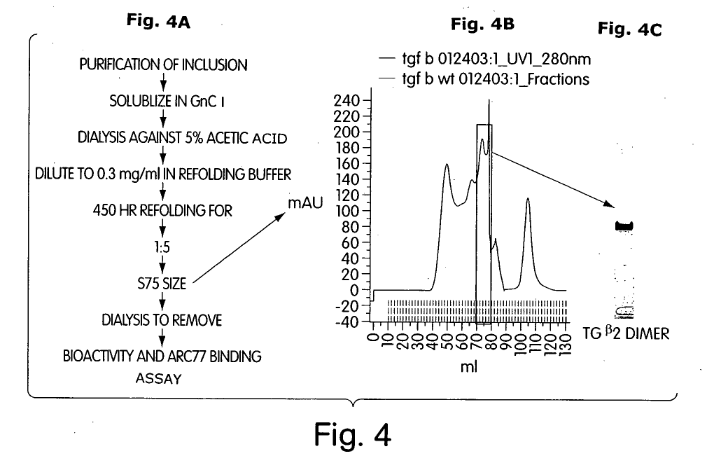

A synthetic polynucleotide encoding full length human TGFβ2 mature protein was cloned into pRSET E. coli expression vector and transformed into BL21 (pLys) strain. The transformed cells were grown under conditions leading to TGFβ2 protein being expressed at high level and forming inclusion bodies. The inclusion bodies were purified and solubilized. The TGFβ2 was refolded and purified by S75 size exclusion chromatography (FIG. 4A). A His-tagged TGFβ2 was also generated by adding His6 plus 30 extra amino acids at the N terminus of TGFβ2. Mutations at positions 59 (S→T), 60 (R→K) and 94 (K→N), referred to herein as the S59T/R60K/K94N mutant, were introduced by site directed mutagenesis. Both the His-tagged and mutated TGFβ2 were expressed, refolded and purified using the same protocol as for wild type TGFβ2. FIG. 4B shows the elution profile of TGFβ2 in S75 size exclusion chromatography, such that the fractions containing the TGFβ2 dimer peaks corresponding to the PAGE band in FIG. 4C are indicated by the box in FIG. 4B.

### Example 2

### Binding of TGFβ2 Protein to TGFβ2-Specific Aptamers

Dissociation constants for the binding of aptamers to purified human or rodent TGFβ-2 protein were determined by nitrocellulose-filter partitioning using ³²P-labeled RNA. In vitro transcribed RNA was treated with calf-intestinal alkaline phophatase (New England Biolabs) to remove 5′-triphosphates, then radio-labeled by incubation with γ-³²P-ATP and T4 polynucleotide kinase (New England Biolabs). Unincorporated label was removed by gel-filtration, and the RNAs were further purified by polyacrylamide gel electrophoresis (PAGE). Purified ³²P-labeled aptamer in water was refolded just prior to use by heating for 3 min at 95° C., followed by a 10 minute room-temperature incubation in binding buffer (50 mM Hepes, pH 7.4, 1 mM MgCl₂, 1 mM CaCl₂, 3 mM KCl, 140 mM NaCl, 0.1 mg/ml BSA, 0.01 mg/ml tRNA). The binding reactions (100 μL) were initiated by the addition of aptamer (≦0.1 nM) to excess TGFβ-2 protein (0.2-100 nM), followed by 10-30 minute equilibration at room temperature.

Nitrocellulose-filter partitioning was performed on Minifold® I, 96-well Dot-Blot manifolds (Schleicher & Schuell). Protein-bound aptamer and residual free aptamer were captured, respectively, on pre-wetted Protran nitrocellulose (Schleicher & Schuell) and Hybond-P polyvinylidene difluoride (Amersham Biosciences) filters by vacuum aspiration, and quantified by PhosphorImager (Amersham Biosciences). The proportion of protein-bound aptamer at each TGFβ-2 concentration was plotted as the ratio of counts-per-minute on the Protran nitrocellulose filter (CPM_{NC}) to the sum of CPM on the Protran and Hybond-P filters (CPMₜₒₜₐₗ). Estimates of dissociation constants (K_{D}) were obtained from a fit of the concentration of TGFβ-2 (i.e., [TGFβ-2])-dependent data to a standard binding isotherm: CPM_{NC}/CPMₜₒₜₐₗ=Cₘₐₓ/(1+K_{D}/[TGFβ2]ₜₒₜₐₗ), where Cₘₐₓ equals the maximum observed value of CPM_{NC}/CPMₜₒₜₐₗ at saturating [TGFβ-2], likely reflecting the proportion of properly-folded aptamer that is competent to recognize TGFβ-2.

Competition assays. Certain aptamers, such as those modified with high molecular weight PEG moieties, bound non-specifically to nitrocellulose, and thus were not amenable to standard nitrocellulose-filter partitioning assays. These aptamers were assayed by competition with other, well-characterized aptamers such as ARC77.

Aptarner competition reactions were prepared by pre-incubation in binding buffer of ³²P-labeled ARC77 (≦0.1 nM) with increasing concentrations of unlabeled, competitor aptamer (0.05-300 nM). Binding reactions were initiated by the addition of aptamer samples to TGFβ-2 protein, yielding a final protein concentration of 2.5 nM. In the absence of cold competitor, ˜30% of ³²P-labeled ARC77 was typically observed to bind to the Protran nitrocellulose membrane at this [TGFβ-2]. The decrease in bound ³²P-labeled ARC77, observed as a function of increasing competitor concentration, was well described by the following model:

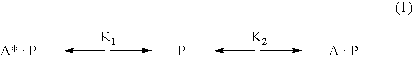

in which A* is ³²P-labeled ARC77, P is TGFβ-2 protein, A is cold competitor aptamer, K₁ is the dissociation constant for the interaction between protein and hot aptamer, and K₂ is the dissociation constant for the interaction between protein and cold competitor aptamer. Estimates of K₂ were obtained from plots of CPM_{NC}/CPMₜₒₜₐₗ versus [cold competitor] ([A]ₜₒₜₐₗ) by fitting the data to Equation 2, derived for this model under the conditions of [A*]<<[P]ₜₒₜₐₗ:

$$A * \cdot P {⟷}^{{K}_{1}} P {⟷}^{{K}_{2}} A \cdot P \qquad ( 1 )$$

where [P●A] is described by the quadratic equation:

$$\frac{{CPM}_{NC}}{{CPM}_{tota}} = \frac{{C}_{max}}{1 + ( 1 + \frac{{[ A ]}_{tota}}{{K}_{2}} - \frac{[ P \cdot A}{{K}_{2}} ) \frac{{K}_{1}}{{[ P ]}_{tota}}} \qquad ( 2 )$$

Each competition assay was accompanied by an independent measurement of K₁, determined by the standard binding assay described above, which was included in the fit.

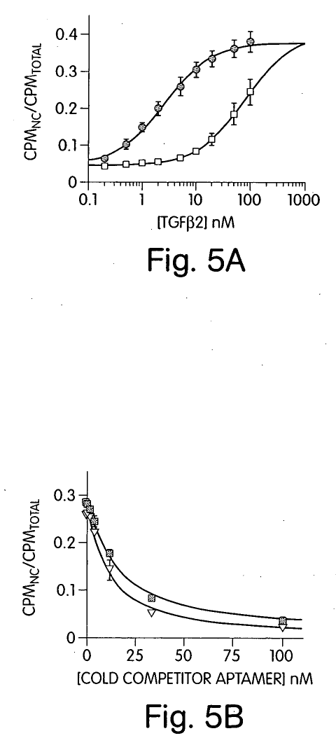

FIGS. 5A and 5B show the binding of TGFβ-2 protein to varying concentrations of TGFβ-2 specific aptamers. (A) ³²P-labeled ARC77 (≦0.1 nM) was incubated with increasing concentrations (0.2-100 nM) of human (●) or rat (□) TGFβ-2 protein and binding was analyzed by nitrocellulose-filter partitioning. The K_{d} values for these aptamers were: ARC77: 3.6 +/−0.6; ARC78: 4.0+/−0.5, and for ARC81:5.1+/−0.4. Estimates of K_{d} were obtained by a fit of the data to Equation 1. (B) Alternatively, estimates of aptamer dissociation constants were obtained by competition with ³²P-labeled ARC77 for binding to human TGFβ-2. Results (shown in FIG. 5B) of the competitive binding of non-radiolabeled ARC77 (∇) and ARC81 (▪) were obtained by a fit of the data to Equation 2 above.

### Example 3

### Species Specific Binding of TGFβ2 Aptamers

Three aptamer compositions of the present invention, ARC77, ARC78 and ARC81, were compared for species binding specificity to human and rodent TGFβ2 and binding affinity according to the methods of Example 2.

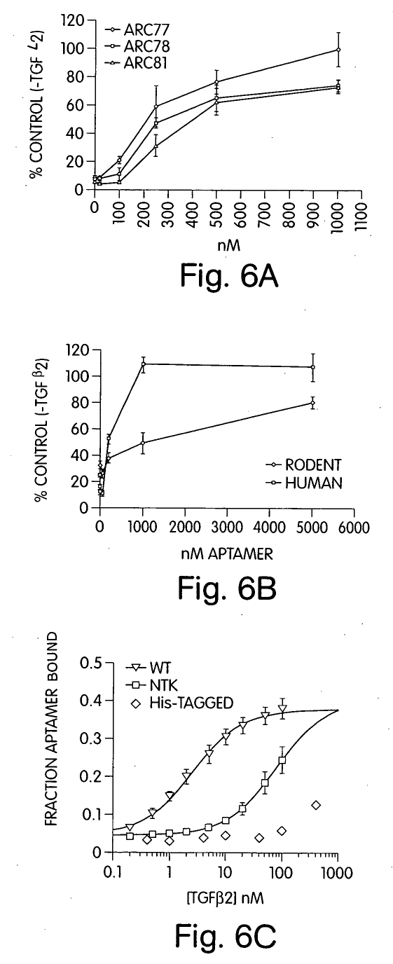

As seen in FIGS. 6A and 6B, the ARC81 aptamer reversed human TGFβ2-mediated inhibition of MLEC proliferation. FIG. 6A shows that the ARC77, ARC78 and ARC81 aptamers inhibit the antiproliferative effects of 50 pg/ml of TGFβ2. An anti-TGFβ2 antibody (R&D, AF-302-NA), which was included as a control, also reversed the effect of low concentrations of aqueous humor on cell proliferation. FIG. 6B shows that the ARC77 aptamer is more potent against the human form of TGFβ2 than the rodent version. Taken together, these data indicate that the ARC77, ARC78 and ARC81 aptamers are able to reverse the biological activity of TGFβ2 as measured using cell proliferation. The ARC77 aptamer also demonstrated specificity against the human versions of TGFβ2 versus the rodent. FIG. 6C shows that the ARC77 aptamer has different binding affinity to human wild type (WT), mouse (NTK) and N-terminal His tagged versions of human TGFβ2: wild type (WT) 2.5±0.3 nM, mouse (NTK) 80±5 nM, and His-tagged ≧500 nM.

MLECs were plated at 2,000 cells per well and incubated at 37° C. for 4 hours. Aptamer and TGFβ2 were added at the indicated concentrations for 16 hours at 37° C. Cell proliferation was measured using BrdU incorporation. BrdU assay was performed as recommended by the manufacturer (Roche Diagnostics).

### Example 4

### Aqueous Humor Challenge Assay

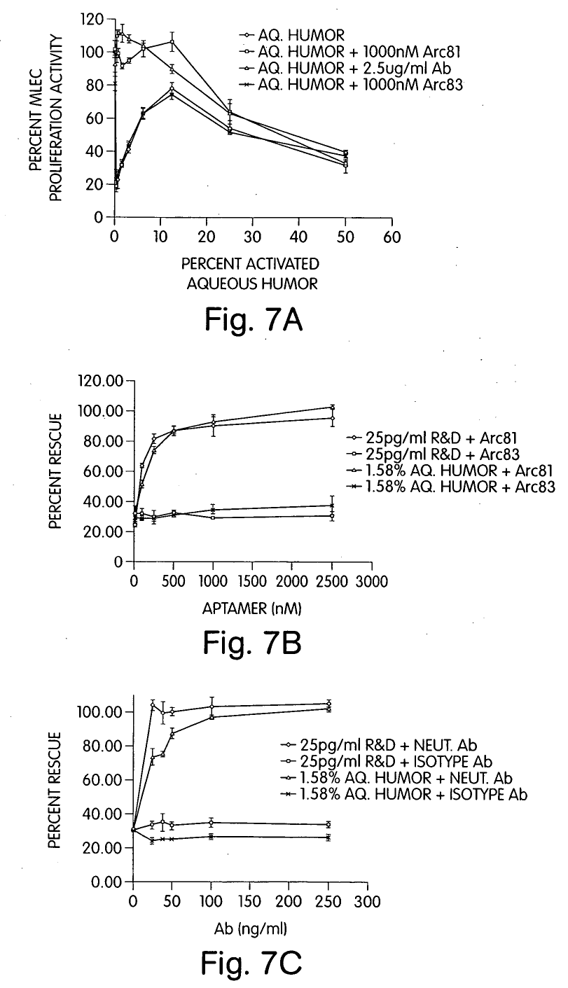

ARC81 reverses aqueous humor-mediated inhibition of MLEC proliferation. FIG. 7A shows that 1000 nM ARC81 inhibited the antiproliferative effects of low concentrations of rabbit aqueous humor (e.g., <10%). An anti-TGFβ2 antibody (R&D, AF-302-NA), which was included as a control, also reversed the effect of low concentrations of aqueous humor on cell proliferation. FIGS. 7B and 7C show that the ARC81 antibody and anti-TGFβ2 antibody rescued rabbit aqueous humor-mediated inhibition of MLEC proliferation in a dose dependent manner. Taken together, these data indicate that the ARC81 aptamer is able to reverse the biological activity of TGFβ2 in aqueous humor.

MLEC Assay. The mink lung epithelial cell proliferation assay is performed over two days. On day 1:1) media is aspirated off from Mink Lung Epithelial Cells (MLEC); 2) the MLEC are washed with 10 ml 1×PBS; 3) 3 ml Trypsin is added and Trypsinize for 3 min at 37° C.; 4) quench with 10 ml 0.5% FBS media; 5) spin at 1000 rpm for 3.30 min; 6) aspirate off supernatant; 7) resuspend pellet in 10 ml 0.5% FBS media; 8) Count 10 μl cell suspension; 9) adjust cell density to 80,000 cells/ml; 10) add 50 μl of cells/well to black bottomed 96-well plates (4000 cells/well); pipet up and down during plating process to avoid settling of cells and uneven plating; 11) incubate cells at 37° C. in 5% CO₂ for 4 hrs to allow adherence of cells; 12) add 25 μl of aptamer (or media, or other test reagent, such as for example, an antibody), preferably the outer wells are not used for treated cells; 13) add 25 μl of TGFβ2 (typically 25 pg/ml); and 14) incubate cells at 37° C. in 5% CO₂ overnight.

On day 2:1) mix 20 μl BrdU with 2 ml 0.5% FBS media; 2) add 10 μl of BrdU mixture/well; 3) incubate cells at 37° C. in 5% CO₂ for 3 hrs; 4) remove media and blot plates dry on paper towels; 5) add 200 μl/well FixDenat solution and incubate for 30 min at room temperature (RT); 6) remove FixDenat solution and blot plates dry on paper towels; 7) add 100 μl /well anti-BrdU POD solution and incubate for 90 min at RT; 8) remove anti-BrdU POD solution and blot plates dry on paper towels; 9) wash plates 3× with 2001 μl/well washing solution with 5 min RT incubations and blot plates dry on paper towels; 10) add 100 μl /well substrate solution and incubate for 3 min at RT in dark; and 11) read plates using PSP Luciferace 1 program on TopCount (Packard Bioscience Co., Downers Grove, Ill.).

MLECs were plated at 2,000 cells per well and incubated at 37° C. for 4 hours. Aptamer, antibody, aqueous humor and TGFβ2 were added at the indicated concentrations for 16 hours at 37° C. Cell proliferation was measured using BrdU incorporation. BrdU assay was performed as recommended by the manufacturer (Roche Diagnostics).

### Example 5

### Selection Minimization and Characterization of TGFβ2-Specific Aptamers

The modified RNA aptamers of the present invention, e.g., ARC77 (SEQ ID No.1) bind native human TGFβ2 and are capable of blocking TGFβ2 effect in Mink Lung Epithelial Cell (MLEC) inhibition assay. For further biochemical characterization of the aptamers, two forms of mature TGFβ2 were generated, native and N-terminal his-tagged versions, in E. coli. After refolding and purification, functional TGFβ2s were obtained. These TGFβ2 were active in cell based assay. N-terminal tags affected both activity and aptamer binding, while the affinity to aptamer was decreased to much larger extent. Further, two mutant TGFβ2s (labeled K94N and S59T/R60K/K94N) were generated based on known isoforms of TGFβ2. The K94N mutant was capable of binding to the aptamers with comparable affinity with that of native TGFβ2, whereas the S59T/R60K/K94N mutant had significantly reduced affinity to the aptamers. Similarly, the aptamers blocked the bioactivity of native and K94N TGFβ2s with higher potencies than that of S59T/R60K/K94N mutant in a cell based assay. Based on a published crystal structure, two substitutions at positions 59 and 60 reside near the dimer interface and adjacent to the N-terminus of TGFβ2, and the other substitution at position 94 is near the type II receptor binding site. Binding competition assay with soluble TGF-b receptors revealed that type III receptor competes with the aptamer binding, but not type II receptor. The data demonstrated that two aptamers bind to one dimer TGFβ2 and that the aptamers of the present invention bind TGFβ2 near or at the TGF-b type III receptor binding site and block its biological function.

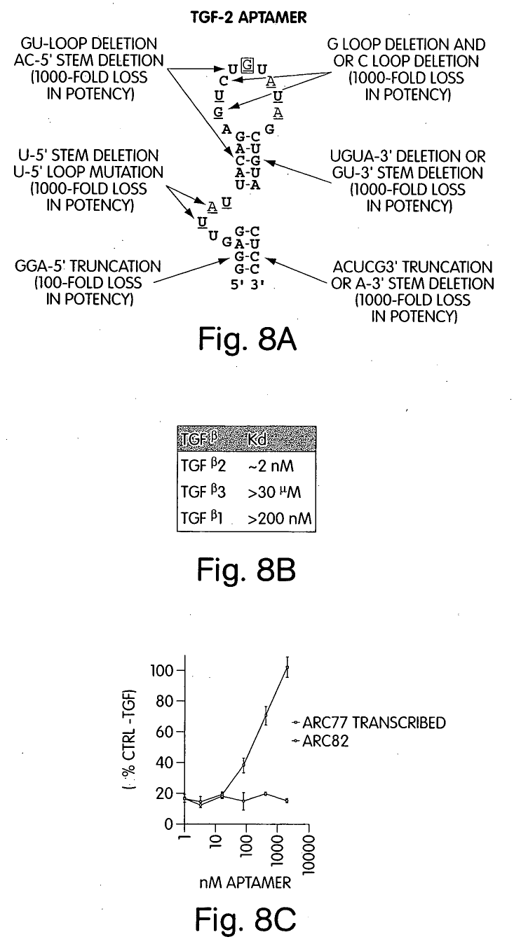

Minimization and mutagenesis/modification analysis of the aptamer. FIG. 8A is an illustration of the selection, minimization and characterization of TGFβ2 aptamers of SEQ ID No. 1 (ARC77). Deletions of residue(s) and the resulting effect on binding affinity are indicated in the text of FIG. 8A and in the table of FIG. 8B. The boxed residues in FIG. 8A represent highly conserved residues. The binding affinity of the aptamer to TGF βs was determined by dot blot protein binding assay (FIG. 8B). TGFβ2 aptamer can reverse the inhibitory effect of TGFβ2 on MLEC cell proliferation. Scrambled aptamer (labeled as “ARC77 transcribed” in FIG. 8C) was used as a negative control, while a TGFβ2 neutralizing antibody was used as a positive control (not shown).

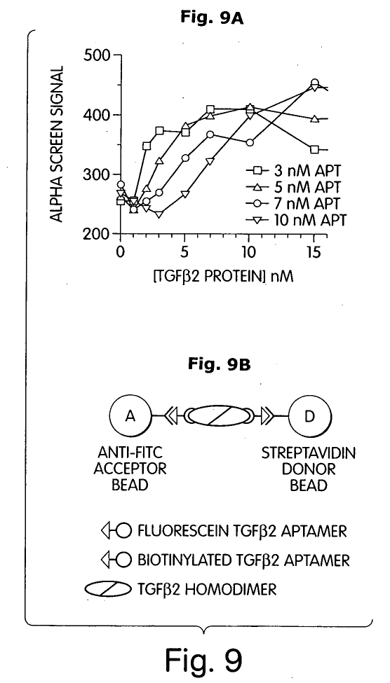

FIG. 9 shows the stoichiometry of the aptamer/TGFβ2 dimer as determined by a screen. Different concentration of the aptamers, labeled by either fluorescein or biotin were bound to anti-FITC acceptor beads or streptavidin donor beads and titrated with TGFβ2 homodimer. The signal was detected with the Fusion plate reader (Packard Bioscience Co., Downers Grove, Ill.).

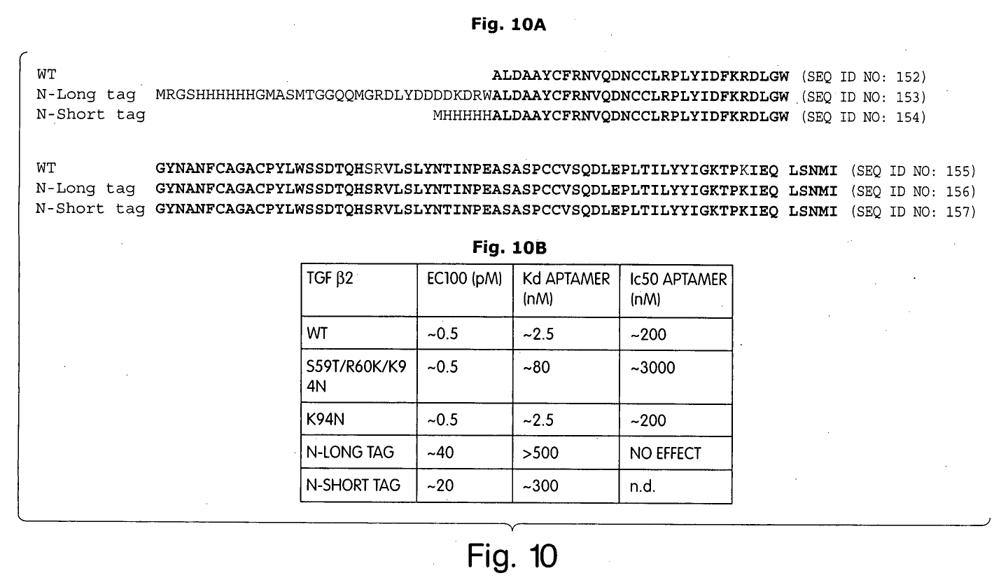

FIG. 10A shows mapping of the aptamer binding site of TGFβ2 by determining the effect of various modifications and/or mutations to wild typeTGFβ2. Three human TGFβ2 variants were tested: wild type TGFβ2, a long tag form of TGFβ2 and a short tag form of TGFβ2. In addition, two mutants were tested. The K94N and S59T/R60K/K94N mutations were introduced into wild type TGFβ2 by quick change site-directed mutagenesis. Each of these proteins (i.e., wild type, S59T;R60K/K94N, K94N, N-long tag TGFβ2 and N-short tag TGFβ2) were incubated in the presence of a TGFb2 aptamer, and their EC100 values, binding affinities and IC50 values (in nM) were determined (FIG. 10B). Binding affinity of the aptamer to those proteins were determined by dot blot. IC50 values of the inhibitory activity of the aptamer were determined using the MLEC proliferation assay.

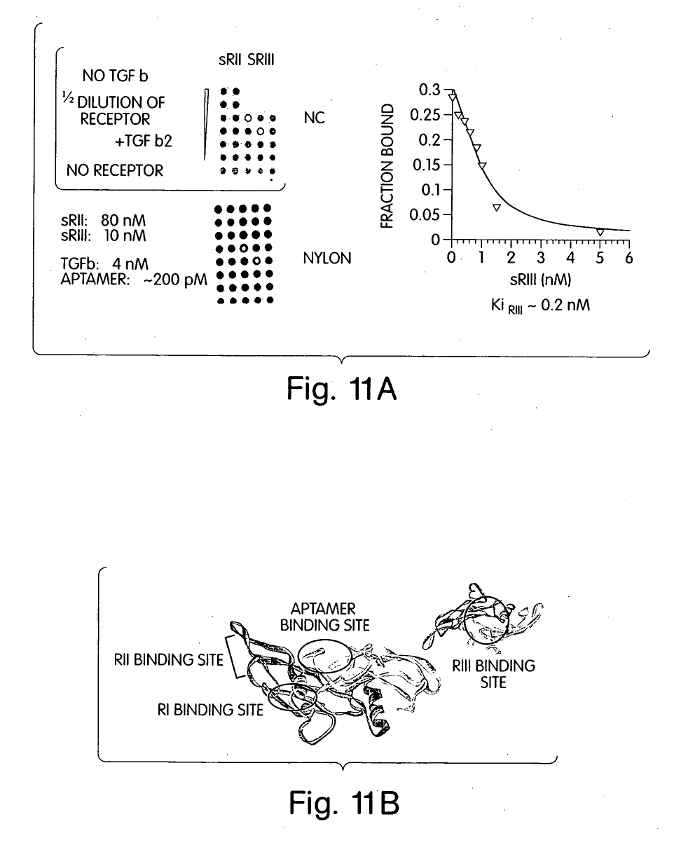

FIG. 11 shows that the TGF type III receptor can block the binding of the aptamer with TGFβ2. Dot blot assays to determine aptamer binding with TGFβ2 were performed after preincubating TGFβ2 with either soluble type III or type II receptors. The Ki was calculated by fitting the data with a simple competition model.

TABLE 1
Aptamer Sequences

SEQ ID No.1 ARC77 - TGFβ2
5′-GGAGGfUfUAfUfUAfCAGAGfUfCfUGfUAfUAGfCfUGfUAfCf
UfCfC-3T-3′
SEQ ID No.2 ARC78 - TGFβ3
5′-NH2-GGAGGfUfUAfUfUAfCAGAGfUfCfUGfUAfUAGfCfUGfU
AfCfUfCfC-3T-3′
SEQ ID No.3 ARC79 - TGFβ2
5′-mGmGmAmGmGfUfUAfUfUmAfCmAmGmAmGfUfCfUGfUAfUAmG
fCfUmGfUmAfCfUfCfc-3T-3′
SEQ ID No.4 ARC81 - TGFβ2
5′-NH2-mGmGmGmGfUfUAfUfUmAfCmAmGmAmGfUfCfUGfUAfUAm
GfCfUmGfUmAfCfCfC-3T-3′
SEQ ID No.5 ARC82 - TGFβ2
5′-mGGmGmGfUfUmAfUfUAfCAmGmAmGfUfCfUmGfUmAfUmAmGf
CfUmGfUAfCfCfC-3T-3′
SEQ ID No.6 ARC111 - TGFβ2
5′-[20KPEG]-NH2-GGAGGfUfUAfUfUAfCAGAGfUfCfUGfUAfUA
GfCfUGfUAfCfUfCfC-3T-3′
SEQ ID No.7 ARC112 - TGFβ2
5′-[PEG30K]-NH2-GGAGGfUfUAfUfUAfCAGAGfUfCfUGfUAfUA
GfCfUGfUAfCfUfCfC-3T-3′
SEQ ID No.8 ARC113 - TGFβ2
5′-[PEG40K]-NH2-GGAGGfUfUAfUfUAfCAGAGfUfCfUGfUAfUA
GfCfUGfUAfCfUfCfC-3T-3′
SEQ ID No.9 ARC117 - TGFβ2
5′-[PEG20K]-NH2-mGmGmGmGfUfUAfUfUmAfCmAmGmAmGfUfCf
UGfUAfUAmGfCfUmGfUmAfCfCfC-3T-3′
SEQ ID No.10 ARC118 - TGFβ2
5′-[PEG30K]-NH2-mGmGmGmGfUfUAfUfUmAfCmAmGmAmGfUfCf
UGfUAfUAmGfCfUmGfUmAfCfCfC-3T-3′
SEQ ID No.11 ARC119 - TGFβ2
5′-[PEG30K]-NH2-mGmGmGmGfUfUAfUfUmAfCmAmGmAmGfUfCf
UGfUAfUAmGfCfUmGfUmAfCfCfC-3T-3′
SEQ ID No.12 ARC120 - TGFβ2
5′-[PEG20K]-NH2-mGGmGmGfUfUmAfUfAfCAmGmAmGfUfCfUmG
fUmAfUmAmGfCfUmGfUAfCfCfC-3T-3′
SEQ ID No.13 ARC121 - TGFβ2
5′-[PEG30K]-NH2-mGGmGmGfUfUmAfUfUAfCAmGmAmGfUfCfUm
GfUmAfUmAmGfCfUmGfUAfCfCfC-3T-3′
SEQ ID No.14 ARC122 - TGFβ2
5′-[PEG40K]-NH2-mGGmGmGfUfUmAfUfUAfCAmGmAmGfUfCfUm
GfUmAfUmAmGfCfUmGfUAfCfCfC-3T-3′
SEQ ID No.21 ARC152 - TGFβ2
5′-[NH2]-mGmGmAmGmGfUfUAFUfUmAfCmAmGmAmGfUfCfUGfUA
fUAmGfCfUmGfUmAfCfUfCfC-3T-3′
SEQ ID No.4 ARC154 - TGFβ2
5′-[NH2]-mGmGmAmGmGfUfUAfUfUmAfCmAmGmAmGfUfCfUGfUA
fUAmGfCfUmGfUmAfCfUfCfC-3T-3′
SEQ ID No.23 ARC155 - TGFβ2
5′C-[NH2]-mGGmGmGfUfUmAfUfUAfCAmGmAmGfUfCfUmGfUmAf
UmAmGfCfUmGfUAfCfCfC-3T-3′
SEQ ID No.24 ARC156 - TGFβ2
5′-[tatp]-[NH2]-mGGmGmGfUfUmAfUfUAfCAmGmAmGfUfCfUm
GfUmAfUmAmGfCfUmGfUAfCfCfC-3T3′
SEQ ID No.25 ARC157 - TGFβ2
5′-[antp]-[NH2]-mGGmGmGfUfUmAfUfUAfCAmGmAmGfUfCfUm
GfUmAfUmAmGfCfUmGfUAfCfCfC-3T-3′
SEQ ID No.26 ARC158 - TGFβ32
5′-[arg7]-[NH2]-mGGmGmGfUfUmAfUfUAfCAmGmAmGfUfCfUm
GfUmAfUmAmGfCfUmGfUAfCfCfC-3T-3′
SEQ ID No.27 ARC159 - TGFβ2
5′-[NH2]-[NH2]-mGmGmAmGmGmUmUmAmUmUmAmCmAmGmAmGmUm
CmUmGmUmAmUmAmGmCmUmGmUmAmCmUmCmC-3T-3′

### Example 6

### PDGF Aptamers in Ocular Disease Treatment

Platelet derived growth factor (PDGF) is a strong mitogen and is known to play a crucial role in a variety of proliferative diseases. Table 1 below shows the dosing concentrations for three sample aptamers useful in ocular disease treatment tested in mice. The dose for administration to young male mice 5-6 weeks old via intravenous is 1 mg/kg or subcutaneous administration is 1, 5, and 20 mg/kg. Timepoints taken after intravenous administration are 0, 5, 10, 20, 40 min, 1, 2, 4, 6, 8, and 10 hours. Timepoints taken after subcutaneous administration are 0, 10, 20, 40 min, 1, 2, 4, 6, 8, 10, and 12 hrs. The binding affinity for ARC125 (SEQ ID No. 16) and ARC127 (PEG-SEQ ID NO:19-PEG-SEQ ID NO:35-PEG-SEQ ID NO:36-3T) bind PDGF AB and BB with a Kd of 100 pM. ARC127 (PEG-SEQ ID NO:19-PEG-SEQ ID NO:35-PEG-SEQ ID NO:36-3T) has been selected from a ssDNA pool and later modified with 2′-O-methyl (underlined) and 2′-fluoro (italicized) and carries 40K PEG group. The 2′-O-methyl, 2′-fluor modifications stabilize the aptamer against nucleases and increase its half life in vivo. The 3′-3′-dT cap increases exonuclease resistance. A 40-K PEG group increases the PK properties of ARC126.

TABLE 2
PDGF specific Aptamers

| | | | Dose | Dose |
|---|---|---|---|---|
| | Mol wt | Ext coeff | concentration² | level³ |
| Sample | (g mol⁻¹) | (L mol⁻¹ cm⁻¹) | (mg ml⁻¹) | (mg eye⁻¹) |
| ARC126 | 10,129.47 | 265,400.00 | 20 | 0.02 |
| ARC127 | 50,128.47 | 265,400.00 | 20 | 0.02 |
| ARC128 | 50,128.47 | 264,800.00 | 20 | 0.02 |

¹as determined by anion exchange HPLC and/or CGE
^{2,3}calculated using aptamer weight only

ARC123 (SEQ ID No. 15), ARC124 (SEQ ID No. 16) and ARC125 (SEQ ID No. 17) have been selected from a ssDNA pool to bind PDGF AB and BB receptors with a K_{d} of 100 pM. They do not have any modified groups but have a 3′-3′-dT cap to increase exonuclease resistance.

TABLE 3

PDGf Aptamers
ARC123 (SEQ ID No.15):
5′-TdGdGdGdAdGdGdGdCdGdCdGTTdCTTdCdGTdGdGTTdAdCTTTTdAdGTdCdCdCd
G-3T-3′
ARC 124 (SEQ ID No.16):
5′-dCdAdCdAdGdGdCTdAdCdGdGdCdAdCdGTdAdGdAdGdCdATdCdAdCdCdATdGdA
TdCdCTdGTdG-3T-3′
ARC125 (SEQ ID No.17):
5′-TdAdCTdCdAdGdGdGdCdAdCTdGdCdAdAdGdCdAdATTdGTdGdGTdCdCdCdAdAT
dGdGdGdCTdGdAdGTdA-3T-3′
ARC126 (SEQ ID NO:18 - PEG - SEQ ID NO:33 - PEG - SEQ ID NO:34)
(functional aptamer):

| | |
|---|---|
| 5′-[NH2]-dCdAdGdGdCfUdAfCmG | (SEQ ID NO:18) - |
| PEG-dCdGTdAmGdAmGdCdAfUfCmA | (SEQ ID NO:33) - |
| PEG-TdGdATfCfCfUmG-3T-3′ | (SEQ ID NO:34) |

ARC127 (PEG - SEQ ID NO. 19 - PEG - SEQ ID NO:35 - PEG -
SEQ ID NO:36 - 3T)
(PEGylated functional aptamer):

| | |
|---|---|
| 5′-[PEG40K]-NH2-dCdAdGdGdCfUdAfCmG | (SEQ ID NO:19) - |
| PEG-dCdGTdAmGdAmGdCdAfUfCmA | (SEQ ID NO:35) - |
| PEG-TdGdATfCfCfUmG-3T-3′ | (SEQ ID NO:36) |

ARC128 (PEG - SEQ ID No.20 - PEG - SEQ ID NO:37 -
PEG - SEQ ID NO:38 - 3T)
(scrambled control):

| | |
|---|---|
| 5′-[PEG40K]-NH2-dCdAdGfCmGfUdAfCmG | (SEQ ID NO:20) - |
| PEG-dCdGTdAdCdCmGdATfUfCmA | (SEQ ID NO:37) - |
| PEG-TdGdAdAdGfCfUmG-3T-3′ | (SEQ ID NO:38) |

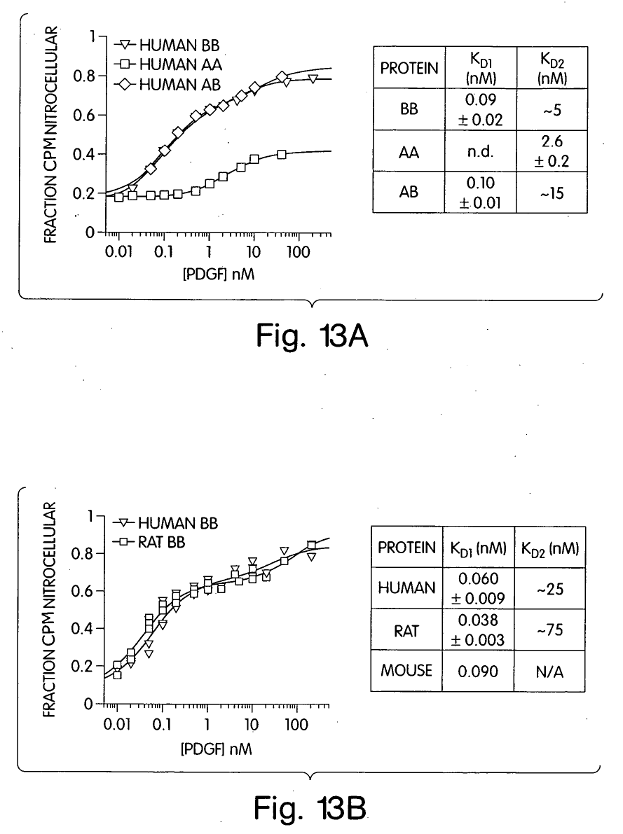

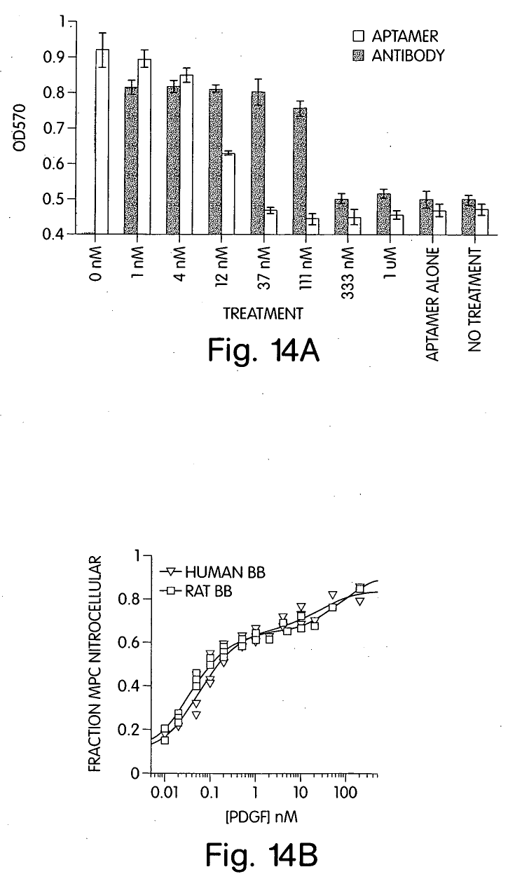

FIGS. 13A and 13B show the binding curves and K_{d} values for ARC127 (PEG-SEQ ID NO:19-PEG-SEQ ID NO:35-PEG-SEQ ID NO:36-3T) demonstrating that ARC127 recognizes the BB and AB isoforms of PDGF but not the AA isoform alone. FIG. 14A and 14B show that ARC127 (PEG-SEQ ID NO:19-PEG-SEQ ID NO:35-PEG-SEQ ID NO:36-3T) binds to human and rat PDGFs with equivalent affinity, and furthermore, the ability of ARC127 to block PDGF-induced 3T3 cell proliferation is comparable to the ability of an anti-PDGF antibody (Upstate/Cell Signaling Solutions) to block PDGF-induced 3T3 cell proliferation.

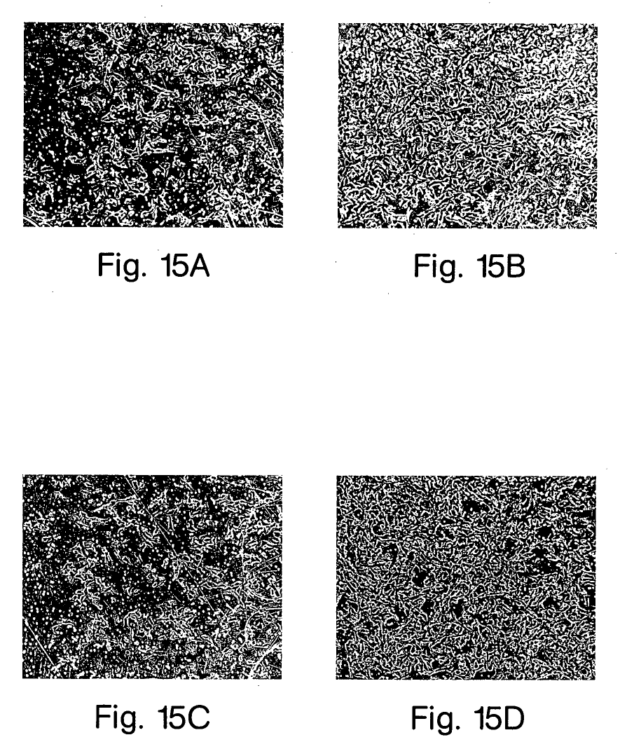

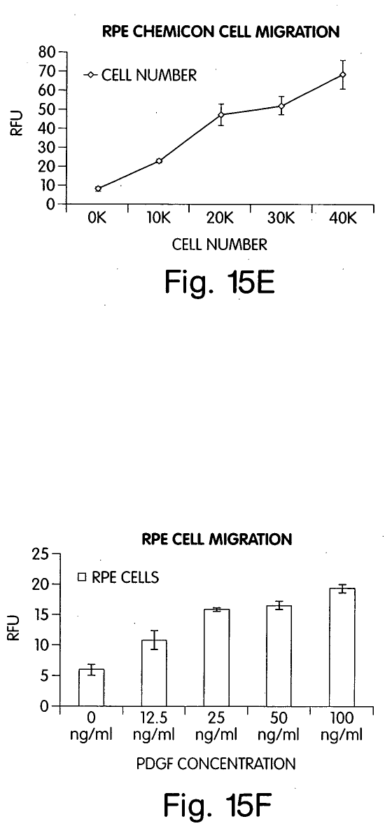

The series of images presented in FIG. 15 show that ARC127 (PEG-SEQ ID NO:19-PEG-SEQ ID NO:35-PEG-SEQ ID NO:36-3T) specifically blocks migration of retinal pigmented epithelial (RPE) cells, while ARC128 (PEG-SEQ ID No. 20-PEG-SEQ ID NO:37-PEG-SEQ ID NO:38-3T), a scrambled aptamer used as a control, has no activity. In particular, FIG. 15A shows the migration of RPE cells with no PDGF. FIG. 15B shows the migration of RPE cells with 100 ng/ml PDGF. FIG. 15C, which shows the migration of RPE cells with PDGF and 100 mM ARC127, demonstrates the blocking effect of ARC127. FIG. 15D, which shows the migration of RPE cells with PDGF and 100 mM ARC128, demonstrates no blocking effect by the scrambled aptamer control (i.e., ARC128). The graphs presented in FIG. 15E and 15F show the increasing effect of PDGF concentration on RPE cell migration.

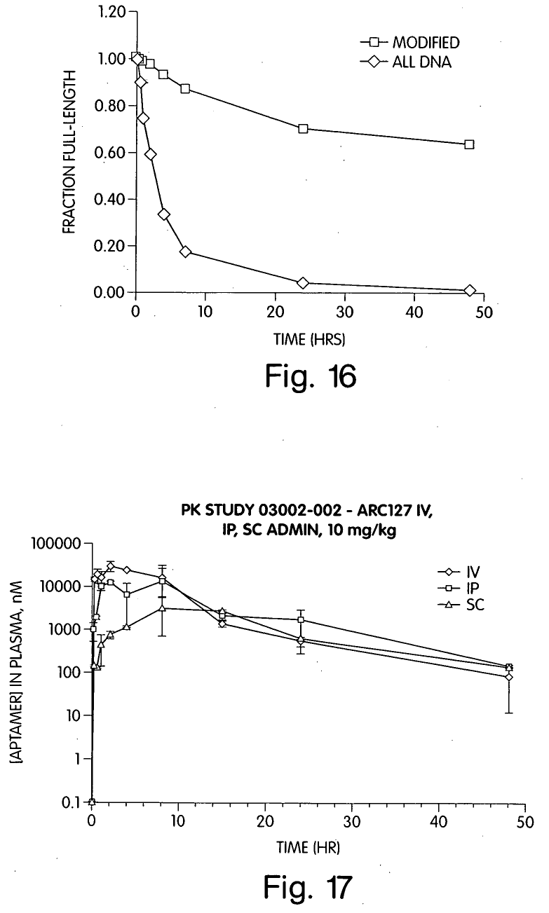

FIG. 16 shows in vitro plasma stability of ARC127 (PEG-SEQ ID NO:19-PEG-SEQ ID NO:35-PEG-SEQ ID NO:36-3T) in 95% plasma at 37° C. The half life (t_{1/2}) for the modified ARC127 was 14 times greater than the t_{1/2} of an all-DNA construct.

### Example 7

### Pharmacokinetic and Bioactivity Profile of ARC127

A pharmacokinetic study (03002-002) was performed to determine the pharmacokinetics of ARC127 via intravenous (IV), intraperitoneal (IP), and subcutaneous (SC) administration in mice. Table 4 below shows the results of the study at a dose of 10 mg/kg showing that bioavailability for intraperitoneal and subcutaneous administration is high with IV, IP, and SC. FIG. 17 shows the concentration of ARC127 aptamer in nM through 50 hours post dose via IV, IP and SC routes of administration. ARC127 was previously found to have the following characteristics: a dissociation constant (K_{d}) of 100 pM; a cellular IC₅₀ value of 2 nM; no observed cytoxicity; efficacy in the animal models for glomerulonephritis, restenosis, cancer and pulmonary hypertension; a Cmax at 1 mg/kg of 2 μM; a solubility of 20 mg/ml; and a systemic half life of 6-12 hours (intravenous injection) and 3.87 days (intravitreal), as shown in Example 12. ARC127 is injectable by various routes of administration, including intravenous, intraperitoneal, subcutaneous and intravitreal. The bioavailability of ARC127 was found to be 62.5% via i.p. injection and 24.0% via subcutaneous injection.

TABLE 4
Pharmacokinetic profile of ARC127 via IV, IP, and SC in
mice at 10 mg/kg.

| | Cmax, | tmax | AUC (hr | MRT | t½ | Vz | bioavailability, |
|---|---|---|---|---|---|---|---|
| | nM | (hr) | nM) | (hr) | (hr) | (L/kg) | F |
| IV | 29711.6 | 2 | 229686.8 | 6.57 | 8.60 | 0.053 | 1.000 |
| IP | 12756.0 | 8 | 143605.5 | 11.23 | 7.86 | 0.078 | 0.625 |
| SC | 3176.7 | 8 | 55030.9 | 16.63 | 9.18 | 0.238 | 0.240 |

As used herein, Cmax refers to the maximum observed serum or plasma concentration; AUC refers to the area under the concentration-time curve; AUClast refers to the area under the concentration-time curve up to the last point in time; AUCinf refers to the area under the concentration-time curve when extrapolated to infinity; T_{1/2} refers to the terminal half life; Cl refers to clearance; MRT refers to the mean residence time; MRTinf refers to mean residence time to infinity; and Vss refers to the apparent volume of distribution.

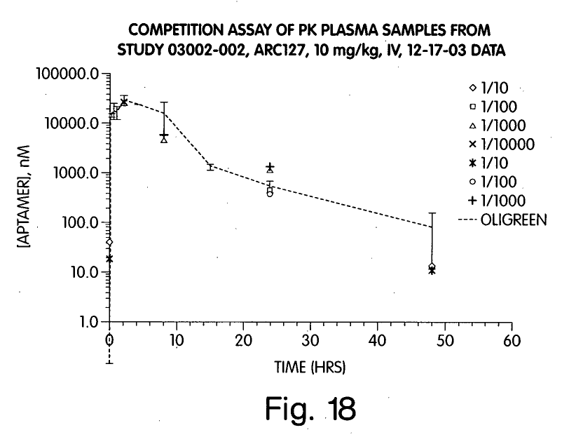

In addition, a second study was performed to determine bioactivity profile of ARC127 post IV administration. The results of the competition binding assay data are consistent with the pharmacokinetic data and show that ARC127 has measurable activity out to 48 hours in vivo (see FIG. 18). These data demonstrate that ARC127 is a potent anti-PDGF aptamer with in vivo efficacy with a K_{d} of 100 pM and a cellular IC₅₀ of 2 nM. In addition, this efficacy has been demonstrated in a number of in vivo models. The pharmacokinetic/pharmacodynamic study shows a systemic half-life of ARC127 between 6-12 hours, and Cₘₐₓ at 1 mg/kg of 2 μM. In addition, ARC127 has shown a half life of 3.5 days in the vitreous humor, as determined using the assay described in Example 4. Taken together, these data indicate that ARC127 is a potent anti-PDGF aptamer, is a novel therapeutic having novel anti-angiogenic properties when co-administered with an anti-VEGF agent, and is useful as a novel oncology agent, as well as a novel therapeutic in PDR and AMD ocular proliferative disease.

### Example 8

### TGFβ2 Therapeutic Aptamers for Intravitreal Administration

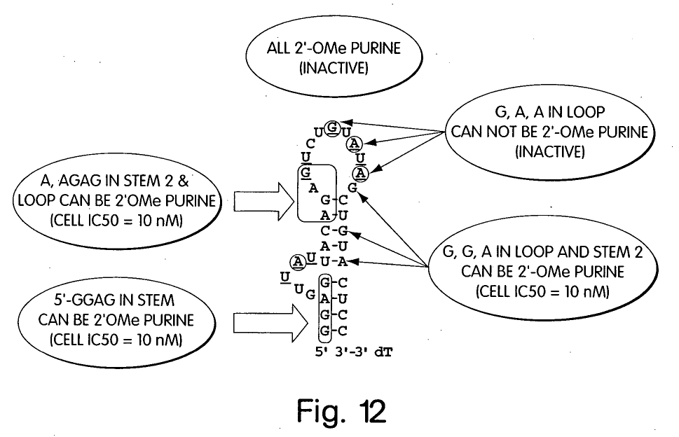

Aptamers with binding affinity to TGFβ2 are administered via intravitreal route with an injected volume of ˜100 μL/eye, via subconjunctival route with an injected volume of ˜250 μL/eye, and via intravenous route with an injected volume ˜250-1000 μL. The dose for each of these routes of administration is 0.5-5 mg/eye for intravitreal administration, 1-5 mg (aptamer mass without conjugation) for subconjunctival administration, and 1-20 mg/kg for intravenous administration. The concentration for each of these routes is 1-5 mg/0.250 ml=4-20 mg/ml for intravitreal administration, 1-5 mg/0.100 ml=10-50 mg/ml for subconjunctival administration, and 1-20 mg/0.250-1.0 ml=1-80 mg/ml for intravenous administration. A biodistribution timecourse is plotted at pre-dose, 5 min, 30 min, 1, 6, 12, 24, and 72 hours for intravitreal and subconjunctival routes of administration, and pre-dose, 5, 30 min, 1, 6, 12, 24, and 48 hours for intravenous route of administration. FIG. 12 shows the configuration of the ARC77 aptamer and illustrates the regions of the aptamer that have been modified.

TABLE 4

TGFβ2 Aptamer sequences
ARC77 SEQ ID No.1: (34 nt; cell IC50 = 10 nM3,
K_{D} = 1 nM) 17, 2′OH purines; 17, 2′F-pyrimidine
5′-G-G-A-G-G-fU-fU-A-fU-fU-A-fC-A-G-A-G-fU-fC-fU-
G-fU-A-fU-A-G-fC-fU-G-fU-A-fC-fU-fC-fC-[3′T];
K_{D} = 1 nM, 9 invariable positions underlined.
ARC79 SEQ ID No.3: (34 nt, cell IC₅₀ = 10 nM,
KD = 1 nM) improved chemical/nuclease stability
through replacement of ribo-residues with 2′-Ome
RNA except 4 essential ribo-nucleotides underlined

### Example 9

### TGFβ2 A Doped Reselection SELEX™ Aptamers

A doped reselection SELEX™ was performed against TGFβ2 using libraries and primers used in Doped re-SELEX :(lower case represent 30% doped residue). These libraries were amplified and transcribed separately and then combined for the initial round:

SEQ ID No.28 TK.82.140.A (14i-1)
TCGGGCGAGTCGTCTGgaaggaattttactacaacgttacttccgcatcc
tccCCGCATCGTCCTCCCTATAGTGAGTCGTATTA
SEQ ID No.29 TK.82.140.B (21a-4)
TCGGGCGAGTCGTCTGgcggacttagtatatacatacgactaaacaacgc
cgcCCGCATCGTCCTCCCTATAGTGAGTCGTATTA
SEQ ID No.30 TK.82.140.C (21a-21)
TCGGGCGAGTCGTCTGggagtacagctatacagactctgtaataacctcc
CCGCATCGTCCTCCCTATAGTGAGTCGTATTA
SEQ ID No.31 5′-Primer TK.82.140.D (sense)
TAATACGACTCACTATAGGGAGGACGATGCGG
SEQ ID No.32 3′-Primer TK.82.140.E (antisense)
TCGGGCGAGTCGTCTG

The doped reselection SELEX™ procedure for the TGFβ2 reselction was performed as follows. For template preparation, the doped DNA libraries were purified using PAGE and amplified by PCR. The purified PCR products were then transcribed with Y639F RNA polymerase in the presence of 2′-F pyrimidine nucleotides, 2′-OH purine ribonucleotides. Resulting RNA pools were used in the first round selection.

The first two rounds of SELEX™ selection were done by nitrocellulose membrane (NC) spot. 500 pmoles human TGFβ2 (hTGFβ2) was spotted on the pre-washed NC membrane and dried by air. The filter then was incubated at room temperature for 1 hour with the combination of 3 RNA doped pools in Dulbecco's phosphate-buffered saline, 1 mM MgCl₂(DPBS). The filter was washed by DPBS 3 times, and the TGFβ2 bound RNAs was eluted in preheated 95C elution buffer (7 M urea 100 mM sodium acetate(pH 5.0) 3 mM EDTA). Eluted RNA was extracted by Phenol/chloroform, precipitated by ethanol and then reverse transcribed, amplified by PCR. The resulting transcription template was transcribed with Y639F single mutant RNA polymerase in the presence of 2′-F pyrimidine nucleotides, 2′-OH purine ribonucleotides and carried to the next round.

SELEX by hydrophobic plate from the third round the SELEX was done by hydrophobic plate selection. 100 μl of 20 nM hTGFβ2 was incubated with NUNC MaxiSorp Plate (positive plate) for 1 hr at 37° C. in DPBS (without 0.1 mg/ml tRNA). In the meantime, RNA pool (with 0.1 mg/ml tRNA ) were incubated in the negative plate for 1 hour at 37° C. for a negative selection. The protein plate was washed 6× with DPBS. The pre-selected RNA pool was incubated in the positive plate for 1 hour at 37° C. The plate was washed 6× with DPBS to remove the unbound RNA. Then, reverse transcription was performed in the plate. The reverse-transcription was then PCR amplified. The resulting transcription template was transcribed with Y639F RNA polymerase in the presence of 2′-F pyrimidine nucleotides, 2′-OH purine ribonucleotides and carried to the next round.

TGFβ2 was serially diluted in DPBS containing 0.2 mg/ml BSA and 0.2 mg/ml tRNA. ³²P-labeled RNA (<20 pM) were incubated with TGFβ2 at room temperature for 30 min. Samples were pipetted with a multichannel pippetor onto a multiwell manifold holding layers of pre-wetted 0.45 micron nitrocellulose, hybond membrane, 3MM filter paper (the order is from top to bottom), aspirated, and washed 3× with DPBS (containing tRNA). All three filters were air dried, exposed to phosphorimager plate, analyzed by ImageQuant. The full length aptamer sequences shown in Table 5 were found to bind to TGFβ2.

TABLE 5
TGFβ2 doped reselection full length aptamer sequences.

| | | |
|---|---|---|
| S5CR12-27 | GGGAGGACGAUGCGGAUCGAGUAUUAUAGAGUAUGUAUAGCUAUACGAUCAGACGACUCGCCCGA | (SEQ ID NO:39) |
| AMX(71)_F7* | GGGAGGACGAUGCGGAUCGAGUAUUAUAGAGUAUGUAUAGCUAUACUAUCAGACGACUCGCCCGA | (SEQ ID NO:40) |
| AMX(71)_A11 | GGGAGGACGAUGCGGAUCGAAUAUUAUAGAGUAUGUAUAGCUAUACGGUCAGACGACUCGCCCGA | (SEQ ID NO:41) |
| AMX(71)_B9 | GGGAGGACGAUGGGGAUCGAGUAUUAUAGAGUCUGUAUAGCUAUACGAUCAGACGACUCGCCCGA | (SEQ ID NO:42) |
| AMX(71)_B11 | GGGAGGACGAUGCGGAUGGAGUAUUAUAGAGUAUGUAUAGCUAUACCAUCAGACGACUCGCCCGA | (SEQ ID NO:43) |
| AMX(71)_C11 | GGGAGGACGAUGCGGAUAGAGCAUUAUAGAGUAUGUAUAGCUAUACUAUCAGACGACUCGCCCGA | (SEQ ID NO:44) |
| ARC232 | | |
| S5CR8-15 | GGGAGGACGAUGCGGAUAGAGUAUUAUAGAGUAUGUAUAGCUAUACUAUCAGACGACUCGCCCGA | (SEQ ID NO:45) |
| AMX(71)_G9 | GGGAGGACGAUGCGGACAGAGUAUUAUAGAGUAUGUGUAGCUAUACCGUCAGACGACUCGCCCGA | (SEQ ID NO:46) |
| S5R12-33 | GGGAGGACGAUGCGGACAGAGUAUUAUAGAGUAUGUAUAGCUAUACUGCCAGACGACUCGCCCGA | (SEQ ID NO:47) |
| S5R12-12 | GGGAGGACGAUGCGGACAGAGUAUUAUAGAGUAUGUAUAGCUAUACUGCAGACGACUCGCCCGA | (SEQ ID NO:48) |
| AMX(71)_F11 | GGGAGGACGAUGCGGACAGAGCAUUAUAGAGUGUGUAUAGCUGUACUGUCAGACGACUCGCCCGA | (SEQ ID NO:49) |
| AMX(71)_F3 | GGGAGGACGAUGCGGACAGAGUAUUAUAGAGUAUGUAUAGCUAUACUAUCAGACGACUCGCCCAA | (SEQ ID NO:50) |
| ARC235 | | |
| S5CR8-45 | GGGAGGACGAUGCGGACAGAGUAUUAUAGAGUAUGUAUAGCUAUACUGUCCAGACGACUCGCCCGA | (SEQ ID NO:51) |
| ARC228 | | |
| S5R8-10 | GGGAGGACGAUGCGGACAGAGUAUUAUAGAGUAUGUAUAGCUAUACUGUCAGACGACUCGCCCGA | (SEQ ID NO:52) |
| AMX(71)_H7 | GGGAGGACGAUGCGGAAAGAGUAUUAUAGAGUCUGUAUAGCUAUACUUUCAGACGACUCGCCCGA | (SEQ ID NO:53) |
| AMX(71)_D10 | GGGAGGACGAUGCGGAANGAAUAUUAUAGAGUAUGUAUAGCUAUACCAUCAGACGACUCGCCCGA | (SEQ ID NO:54) |
| S5CR12-12 | GGGAGGACGAUGCGGAAGGAGUAUUAUAGAGUAUGUAUAGCUAUACCAGACGACUCGCCCGA | (SEQ ID NO:55) |
| S5CR12-15 | GGGAGGACGAUGCGGAAGGAGUAUUAUAGAGUAUGUAUAGCUAUACCAUCAGACGACUCGCCCGA | (SEQ ID NO:56) |
| AMX(71)_H10 | GGGAGGACGAUGCGGAAGGAAUAUUAUAGAGUAUGUAUAGCUAUACCAUCAGACGACUCGCCCGA | (SEQ ID NO:57) |
| ARC233 | | |
| S5CR8-18 | GGGAGGACGAUGCGGAGGGAUUAUUAUAGAGUCUGUAUAGCUAUACCCUCAGACGACUCGCCCGA | (SEQ ID NO:58) |
| AMX(71)_A8 | GGGAGGACGAUGCGGAGGGAAUAUUAUAGAGUAUGUAUAGCUAUACCAUCAGACGACUCGCCCGA | (SEQ ID NO:59) |
| AMX(71)_H9 | GGGAGGACGAUGCGGAGAAGAGUAUUAUAGAGUCUGUAUAGCUAUACUUCAGACGACUCGCCCGA | (SEQ ID NO:60) |
| AMX(74)_G6 | GGGAGGACGAUGCGGAGAGAUUAUUAUAGAGUAUGUAUAGCUGUACUGCCAGACGACUCGCCCGA | (SEQ ID NO:61) |
| ARC231 | | |
| S5CR8-14 | GGGAGGACGAUGCGGAACGAAUAUUACAGAGUAUGUAUAGCUGUACGGUCAGACGACUCGCCCGA | (SEQ ID NO:62) |
| S5CR8-28 | GGGAGGACGAUGCGGAGUGAGUAUUAUAGAGUAUGUAUAGCUAUACACAUCAGACGACUCGCCCGA | (SEQ ID NO:63) |
| AMX(71)_A10 | GGGAGGACGAUGCGGUGUGAAUAUUAUAGAGUCUGUAUAGCUAUACCAUCAGACGACUCGCCCGA | (SEQ ID NO:64) |
| AMX(74)_F1 | GGGAGGACGAUGCGGUGUGAAUAUUAUAGAGUCUGUAUAGCUAUACCACCAGACGACUCGCCCGA | (SEQ ID NO:65) |
| ARC227 | | |
| S5R8-1 | GGGAGGACGAUGCGGUGUGAGUAUUAUAGAGUCUGUAUAGCUAUACCACCAGACGACUCGCCCGA | (SEQ ID NO:66) |
| AMX(71)_B3 | GGGAGGACGAUGCGGUCCGCAUUAUCUUCUACGUUACAUAUACUAUCUCUGUCAGACGACUCGCCCGA | (SEQ ID NO:67) |
| AMX(71)_D11 | GGGAGGACGAUGCGGUCGGAGUAUUAUAGAGUAUGUAUAGCUAAACCGUCAGACGACUCGCCCGA | (SEQ ID NO:68) |
| AMX(74)_A2 | GGGAGGACGAUGCGGUAAGAGUAUUACAGAGUAUGUAUAGCUGUACUUGCCAGACGACUCGCCCGA | (SEQ ID NO:69) |
| AMX(74)_E4 | GGGAGGACGAUGCGGUGAGAAUAUUACAGAGUAUGUAUAGCUGUACUUGCCAGACGACUCGCCCGA | (SEQ ID NO:70) |
| AMX(74)_F4 | GGGAGGACGAUGCGGUGAGAAUAUUAUAGAGUAUGUAUAGCUAUACUCGCCAGACGACUCGCCCAA | (SEQ ID NO:71) |
| AMX(74)_F3 | GGGAGGANGANGCGGUGAGAGUAUUAUAGAGUCUGUAUAGCUAUACUGCCAGACGACUCGCCCGA | (SEQ ID NO:72) |
| ARC229 | | |
| S5R8-43 | GGGAGGACGAUGCGGUGAGAGUAUUAUAGAGUCUGUAUAGCUAUACUCGCCAGACGACUCGCCCGA | (SEQ ID NO:73) |
| ARC234 | | |
| S5CR8-32 | GGGAGGACGAUGCGGUGAGAGUAUUAUAGAGUCUGUAUAGCUAUACCACCAGACGACUCGCCCGA | (SEQ ID NO:74) |
| AMX(71)_D9 | GGGAGGACGAUGCGGUAGAGUAUUAUAGAGUAUGUAUAGCUAUACACUCAGACGACUCGCCCAA | (SEQ ID NO:75) |
| AMX(71)_H12 | GGGAGGACGAUGCGGUAGAGUAUUAGAGAGUAUGUAUAGCUAUACCAUCAGACGACUCGCCCGA | (SEQ ID NO:76) |
| AMX(71)_G7 | GGGAGGACGAUGCGGUGGGAAUAUUAUAGAGUCUGUAUAGCUAUACCCUCAGACGACUCGCCCGA | (SEQ ID NO:77) |
| S5CR12-14 | GGGAGGACGAUGCGGGCGGAAUAUUAUAGAGUAUGGAUAGCUAUACCGUCAGACGACUCGCCCGA | (SEQ ID NO:78) |
| ARC230 | | |
| S5R8-45 | GGGAGGACGAUGCGGGACAGAGUAUUAUAGAGUAUGUAUAGCUAUACUGUCAGACGACUCGCCCGA | (SEQ ID NO:79) |
| AMX(71)_B7 | GGGAGGACGAUGCGGGCAGAGUAUUAUAGAGUACGUAUAGCUAUACUGUCAGACGACUCGCCCGA | (SEQ ID NO:80) |
| AMX(71)_A9 | GGGAGGACGAUGCGGGCAGAGUAUUAUAGAGUAUGUAUAGCUAUACUGUCAGACGACUCGCCCGA | (SEQ ID NO:81) |
| AMX(71)_G8 | GGGAGGACGAUGCGGGUGGAGUAUUAUAGAGUAUGUAUAGCUAUACUAUCAGACGACUCGCCCAA | (SEQ ID NO:82) |
| AMX(74)_A3 | GGGAGGACGAUGCGGGUAGAUUAUUACAGAGUCUGUAUAGCUGUACUGCCAGACGACUCGCCCGA | (SEQ ID NO:83) |
| AMX(74)_E2 | GGGAGGACGAUGCGGGUAGAAUAUUAUAGAGUCUGUAUAGCUAUACUGCCAGACGACUCGCCCGA | (SEQ ID NO:84) |
| AMX(74)_H1 | GGGAGGACGAUGCGGGUAGAAUAUUACAGAGUAUGUAUAGCUGUACUGCCAGACGACUCGCCCGA | (SEQ ID NO:85) |
| AMX(74)_C3 | GGGAGGACGAUGCGGGGAGAAUAUUACAGAGUAUGUAUAGCUGUACUUGCCAGACGACUCGCCCGA | (SEQ ID NO:86) |
| AMX(74)_A1 | GGGAGGACGAUGCGGGGAGAAUAUUACAGAGUAUGUAUAGCUGUACUGCCAGACGACUCGCCCGA | (SEQ ID NO:87) |
| AMX(74)_B3 | GGGAGGACGAUGCGGGGAGAUUAUUAUAGAGUAUGUAUAGCUAUACUGCCAGACGACUCGCCCGA | (SEQ ID NO:88) |
| AMX(74)_G3 | GGGAGGACGAUGCGGGGAGAGUAUUAUAGAGUAUGUAUAGCUAUACUGCCAGACGACUCGCCCGA | (SEQ ID NO:89) |
| AMX(74)_D3 | GGGAGGACGAUGCGGGAAGAGUAUUACAGAGUAUGUAUAGCUGUACUGCCAGACGACUCGCCCGA | (SEQ ID NO:90) |
| AMX(74)_E6 | GGGAGGACGAUGCGGGCAAAGUAUUGUAGAGUAUGCAUAGCUAUAUUGCCAGACGACUCGCCCGA | (SEQ ID NO:91) |
| 21a-21 | GGGAGGACGAUGCGGGGAGGUUAUUACAGAGUCUGUAUAGCUGUACUCCCAGACGACUCGCCCGA | (SEQ ID NO:92) |
| 14i-1 | GGGAGGACGAUGCGGGGAGGAUGCGGAAGUAACGUUGUAGUAAAAUGCCUUCCAGACGACUCGCCCGA | (SEQ ID NO:93) |
| 21a-4 | GGGAGGACGAUGCGGGCGGCGUUGUUUAGUCGUAUGUAUAUACUAAGUCCGCCAGACGACUCGCCCGA | (SEQ ID NO:94) |

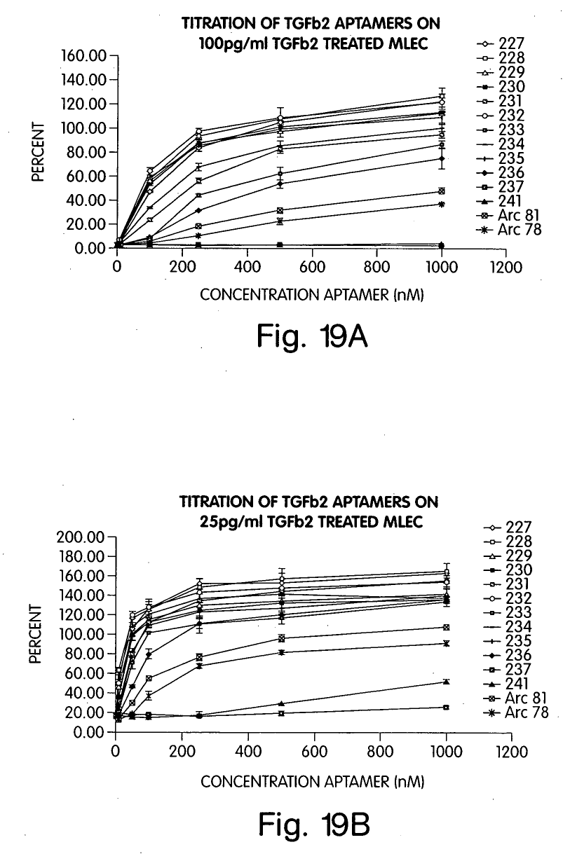

Binding data for the full-length aptamer sequences shown above are shown in Table 6. FIG. 19A and 19B show binding plots for the full-length sequences shown in Table 5.

TABLE 6
Binding data as performed in MLEC inhibition assay
CW115-95, 117, 129

| | | |
|---|---|---|
| 129: | S5R8-1: | 7.4 nM |
| | S5R8-10: | 14.5 nM |
| | S5R8-43: | 6.5 nM |
| | S5R8-45: | 12.1 |
| | S5CR8-15: | 17.9 nM |
| | S5CR8-18: | 5.3 nM |
| | S5CR8-32: | 8.0 nM |
| | S5CR8-35: | 8.4 nM |
| | S5CR8-45: | 5.2 nM |

TABLE 7
Minimized TGFβ2 doped reselection aptamers

| | |
|---|---|
| CW128.10.A(S5CR12-27) | |
| AfUfCGAGfUAfUfUAfUAGAGfUAfUGfUAfUAGfCfUAfUAfCGAfU[3T] | (SEQ ID NO:95) |
| CW128.10.B(AMX(71)_F7) | |
| AfUfCGAGfUAfUfUAfUAGAGfUAfUGfUAfUAGfCfUAfUAfCfUAfU[3T] | (SEQ ID NO:96) |
| CW128.10.C(AMX(71)_A11) | |
| AfUfCGAAfUAfUfUAfUAGAGfUAfUGfUAfUAGfCfUAfUAfCGGfU[3T] | (SEQ ID NO:97) |
| CW128.10.D(AMX(71)_B9) | |
| AfUfCGAGfUAfUfUAfUAGAGfUfCfUGfUAfUAGfCfUAfUAfCGAfU[3T] | (SEQ ID NO:98) |
| ARC285 | |
| 8.10.E(AMX(71)_B11) | |
| AfUGGAGfUAfUfUAfUAGAGfUAfUGfUAfUAGfCfUAfUAfCfCAfU[3T] | (SEQ ID NO:99) |
| CW128.10.F(AMX(71)_C11) | |
| AfUAGAGfCAfUfUAfUAGAGfUAfUGfUAfUAGfCfUAfUAfCfUAfU[3T] | (SEQ ID NO:100) |
| CW128.10.G(S5CR8-15, full length ARC232) | |
| AfUAGAGfUAfUfUAfUAGAGfUAfUGfUAfUAGfCfUAfUAfCfUAfU[3T] | (SEQ ID NO:101) |
| CW128.10.H(AMX(71)_G9) | |
| AfCAGAGfUAfUfUAfUAGAGfUAfUGfUGfUAGfCfUAfUAfCfCGfU[3T] | (SEQ ID NO:102) |
| CW128.10.I(S5R12-33) | |
| AfCAGAGfUAfUfUAfUAGAGfUAfUGfUAfUAGfCfUAfUAfCfUGfC[3T] | (SEQ ID NO:103) |
| CW128.10.J(S5R12-12) | |
| AfCAGAGfUAfUfUAfUAGAGfUAfUGfUAfUAGfCfUAfUAfCfUG[3T] | (SEQ ID NO:104) |
| CW128.10.K(AMX(71)_F11) | |
| AfCAGAGfCAfUfUAfUAGAGfUGfUGfUAfUAGfCfUGfUAfCfUGfU[3T] | (SEQ ID NO:105) |
| CW128.10.L(AMX(71)_F3) | |
| AfCAGAGfUAfUfUAfUAGAGfUAfUGfUAfUAGfCfUAfUAfCfUAfU[3T] | (SEQ ID NO:106) |
| ARC283, | |
| CW128.10.M(S5CR8-45, full length ARC235) | |
| AfCAGAGfUAfUfUAfUAGAGfUAfUGfUAfUAGfCfUAfUAfCfUGfUfC[3T] | (SEQ ID NO:107) |
| ARC286, | |
| CW128.10.N(S5R8-10, full length ARC22B) | |
| AfCAGAGfUAfUfUAfUAGAGfUAfUGfUAfUAGfCfUAfUAfCfUGfU[3T] | (SEQ ID NO:108) |
| CW128.10.O(AβVC(71)_H7) | |
| AAAGAGfUAfUfUAfUAGAGfUfCfUGfUAfUAGfCfUAfUAfCfUfUfU[3T] | (SEQ ID NO:109) |
| CW128.10.P(AMX(71)_D10) | |
| AAIβGAAfUAfUfUAfUAGAGfUAfUGfUAfUAGfCfUAfUAfCfCAfU[3T] | (SEQ ID NO:110) |
| CW128.10.Q(S5CR12-12) | |
| AAGGAGfUAfUfUAfUAGAGfUAfUGfUAfUAGfCfUAfUAfC[3T] | (SEQ ID NO:111) |
| ARC281 | |
| CW128.10.R(S5CR12-15) | |
| AAGGAGfUAfUfUAfUAGAGfUAfUGfUAfUAGfCfUAfUAfCfCAfU[3T] | (SEQ ID NO:112) |
| ARC287 | |
| CW128.10.S(AMX(71)_H10) | |
| AAGGAAfUAfUfUAfUAGAGfUAfUGfUAfUAGfCfUAfUAfCfCAfU[3T] | (SEQ ID NO:113) |
| CW128.10.T(S5CR8-18, full length ARC233) | |
| AAfCGAAfUAfUfUAfCAGAGfUAfUGfUAfUAGfCfUGfUAfCGGfU[3T] | (SEQ ID NO:114) |
| ARC282 | |
| CW128.10.U(AMX(71)_A8) | |
| AGGGAAfUAfUfUAfUAGAGfUAfUGfUAfUAGfCfUAfUAfCfCAfU[3T] | (SEQ ID NO:115) |
| CW128.10.V(AMX(71)_H9) | |
| AGAAGAGfUAfUfUAfUAGAGfUfCfUGfUAfUAGfCfUAfUAfCfUfU[3T] | (SEQ ID NO:116) |
| CW128.10.W(AMX(74)_G6) | |
| AGAGAfUfUAfUfUAfUAGAGfUAfUGfUAfUAGfCfUGfUAfCfUGfC[3T] | (SEQ ID NO:117) |
| CW128.11.A(S5CR8-14, full length ARC231) | |
| AAfCGAAfUAfUfUAfCAGAGfUAfUGfUAfUAGfCfUGfUAfCGGfU[3T] | (SEQ ID NO:118) |
| CW128.11.B (S5CR8-28) | |
| AGfUGAGfUAfUfUAfUAGAGfUAfUGfUAfUAGfCfUAfUAfCAfCAfU[3T] | (SEQ ID NO:119) |
| CW128.11.C(AMX(71)_A10) | |
| fUGfUGAAfUAfUfUAfUAGAGfUfCfUGfUAfUAGfCfUAfUAfCfCAfU[3T] | (SEQ ID NO:120) |
| CW128.11.D(AMX(74)_F1) | |
| fUGfUGAAfUAfUfUAfUAGAGfUfCfUGfUAfUAGfCfUAfUAfCfCAfC[3T] | (SEQ ID NO:121) |
| CW128.11.E(S5R8-1, full length ARC227) | |
| fUGfUGAGfUAfUfUAfUAGAGfUfCfUGfUAfUAGfCfUAfUAfCfCAfC[3T] | (SEQ ID NO:122) |
| CW128.11.F(AMX(71)_B3) | |
| fUfCfCGfCAfUfUAfUfCfUfUfCfUAfCGfUfUAfCAfUAfUAfCfUAfUfCfUfCfUGfU[3T] | (SEQ ID NO:123) |
| CW128.11.G(AMC(71)_D11) | |
| fUfCGGAGfUAfUfUAfUAGAGfUAfUGfUAfUAGfCfUAAAfCfCGfU[3T] | (SEQ ID NO:124) |
| CW128.11.H(AMX(74)_A2) | |
| fUAAGAGfUAfUfUAfCAGAGfUAfUGfUAfUAGfCfUGfUAfCfUfUGfC[3T] | (SEQ ID NO:125) |
| CW128.11.I(AMX(74)_E4) | |
| fUGAGAAfUAfUfUAfCAGAGfUAfUGfUAfUAGfCfUGfUAfCfUfUGfC[3T] | (SEQ ID NO:126) |
| CW128.11.J(AMX(74)_F4) | |
| fUGAGAAfUAfUfUAfUAGAGfUAfUGfUAfUAGfCfUAfUAfCfUfCGfC[3T] | (SEQ ID NO:127) |
| CW128.11.K(AMX(74)_F3) | |
| fUGAGAGfUAfUfUAfUAGAGfUfCfUGfUAfUAGfCfUAfUAfCfUGfC[3T] | (SEQ ID NO:128) |
| CW128.11.L(S5R8-43, full length ARC229) | |
| fUGAGAGfUAfUfUAfUAGAGfUfCfUGfUAfUAGfCfUAfUAfCfUfCGfC[3T] | (SEQ ID NO:129) |
| CW128.11.M(S5CR8-32, full length ARC234) | |
| fUGAGAGfUAfUfUAfUAGAGfUfCfUGfUAfUAGfCfUAfUAfCfCAfC[3T] | (SEQ ID NO:130) |
| CW128.11.N(AMX(71)_D9) | |
| fUAGAGfUAfUfUAfUAGAGfUAfUGfUAfUAGfCfUAfUAfCAfCfU[3T] | (SEQ ID NO:131) |
| CW128.11.O(AMX(71)_H12) | |
| fUAGAGfUAfUfUAfUAGAGfUAfUGfUAfUAGfCfUAfUAfCfCAfU[3T] | (SEQ ID NO:132) |
| CW128.11.P(AMX(71)_G7) | |
| AAfUAfUfUAfUAGAGfUfCfUGfUAfUAGGCfUAfUAfCfCfCfU[3T] | (SEQ ID NO:133) |
| CW128.11.Q (S5CR12-14) | |
| GfCGGAAfUAfUfUAfUAGAGfUAfUGGAfUAGfCfUAfUAfCfCGfU[3T] | (SEQ ID NO:134) |
| ARC284 | |
| CW128.11.R(S5R8-45, full length ARC230) | |
| GAfCAGAGfUAfUfUAfUAGAGfUAfUGfUAfUAGfCfUAfUAfCfUGfU[3T] | (SEQ ID NO:135) |
| CW128.11.S(AMX(71)_B7) | |
| GfCAGAGfUAfUfUAfUAGAGfUAfCGfUAfUAGfCfUAfUAfCfUGfU[3T] | (SEQ ID NO:136) |
| CW128.11.T(AMX(71)_A9) | |
| GfCAGAGfUAfUfUAfUAGAGfUAfUGfUAfUAGfCfUAfUAfCfUGfU[3T] | (SEQ ID NO:137) |
| CW128.11.fU(AMX(71)_G8) | |
| GfUGGAGfUAfUfUAfUAGAGfUAfUGfUAfUAGfCfUAfUAfCfUAfU[3T] | (SEQ ID NO:138) |
| CW128.11.V(AMX(74)_A3) | |
| GfUAGAfUfUAfUfUAfCAGAGfUfCfUGfUAfUAGfCfUGfUAfCfUGfC[3T] | (SEQ ID NO:139) |
| CW128.11.W(AMX(74)_E2) | |
| GfUAGAAfUAfUfUAfUAGAGfUfCfUGfUAfUAGfCfUAfUAfCfUGfC[3T] | (SEQ ID NO:140) |
| CW128.12.A(AMX(74)_H1) | |
| GfUAGAAfUAfUfUAfCAGAGfUAfUGfUAfUAGfCfUGfUAfCfUGfC[3T] | (SEQ ID NO:141) |
| CW128.12.B(AMX(74)_C3) | |
| GGAGAAfUAfUfUAfCAGAGfUAfUGfUAfUAGfCfUGfUAfCfUfUGfC[3T] | (SEQ ID NO:142) |
| CW128.12.C(AMX(74)_A1) | |
| GGAGAAfUAfUfUAfCAGAGfUAfUGfUAfUAGfCfUGfUAfCfUGfC[3T] | (SEQ ID NO:143) |
| CW128.12.D(AMX(74)_B3) | |
| GGAGAfUfUAfUfUAfUAGAGfUAfUGfUAfUAGfCfUAfUAfCfUGfC[3T] | (SEQ ID NO:144) |
| CW128.12.E(AMX(74)_G3) | |
| GGAGAGfUAfUfUAfUAGAGfUAfUGfUAfUAGfCfUAfUAfCfUGfC[3T] | (SEQ ID NO:145) |
| CW128.12.G(AMX(74)_D3) | |
| GAAGAGfUAfUfUAfCAGAGfUAfUGfUAfUAGfCfUGfUAfCfUGfC[3T] | (SEQ ID NO:146) |
| CW128.12.G(AMX(74)_E6) | |
| GfCAAAGfUAfUfUGfUAGAGfUAfUGfCAfUAGfCfUAfUAfUfUGfC[3T] | (SEQ ID NO:147) |
| CW128.12.H(21a-21, full length ARC236) | |
| GGAGGfUfUAfUfUAfCAGAGfUfCfUGfUAfUAGfCfUGfUAfCfUfCfC[3T] | (SEQ ID NO:148) |
| CW128.12.I(14i-1, full length ARC237) | |
| GGAGGAfUGfCGGAAGfUAAfCGfUfUGfUAGfUAAAAfUfUfCfCfUfUfC[3T] | (SEQ ID NO:149) |
| CW128.12.J(21a-4, full length ARC241) | |
| GfCGGfCGfUfUGfUfUfUAGfUfCGfUAfUGfUAfUAfUAfCfUAAGfUfCfCGfC[3T] | (SEQ ID NO:150) |

TABLE 8
Binding information on truncated aptamers

| CW128_103 | ARC235 | ARC236 | ARC281 | ARC282 | ARC283 | ARC284 | CW128.10.F |
|---|---|---|---|---|---|---|---|
| Kd(nM) | 0.53 | 0.75 | 1.42 | 0.62 | 1.27 | 0.11 | 3.66 |

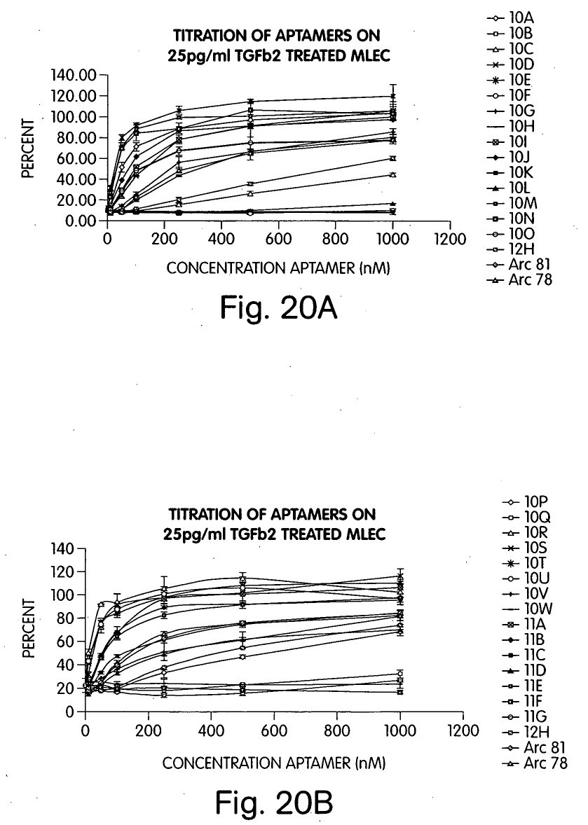

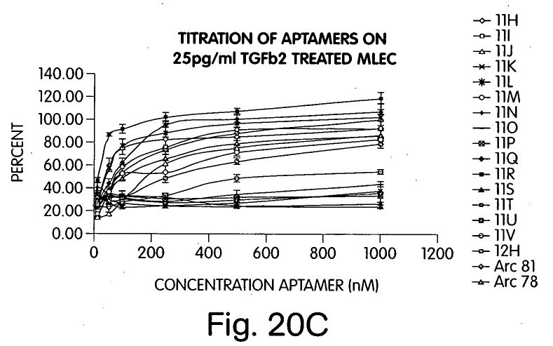

FIGS. 20A, 20B, and 20C show binding plots for the truncated aptamers shown in Table 7.

### Example 10

### Selection Minimization and Characterization of VEGF Receptor 2 (VEGF 42)-Specific Aptamers

Aptamers directed to the VEGF receptor 2 (VEGF R2), also known as the KDR molecule, were isolated using semi-automated SELEX™ procedure. Fifteen rounds of selection were performed over three weeks. 48 clones were identified and grouped into the 9 families. These sequences were analyzed to identify functional motifs. Aptamers directed to the VEGF receptor were found to have a K_{d} in the range of 1-3 nM.

### Example 11

Pharmacokinetic and Bioactivity Profile of ARC81, ARC117 and ARC119

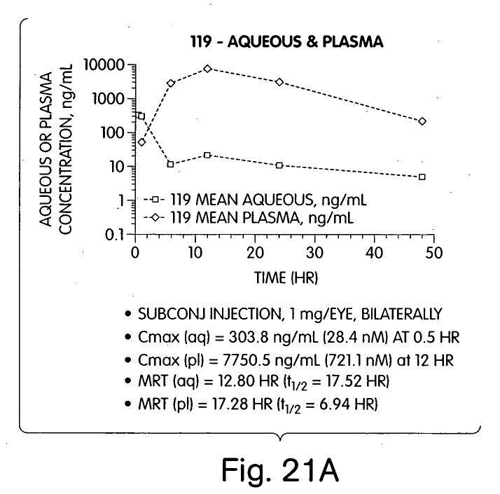

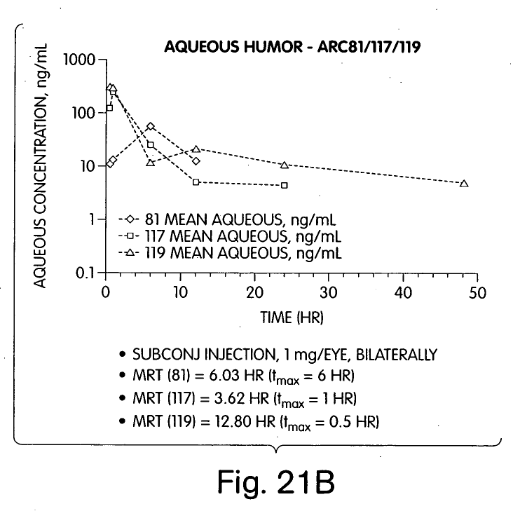

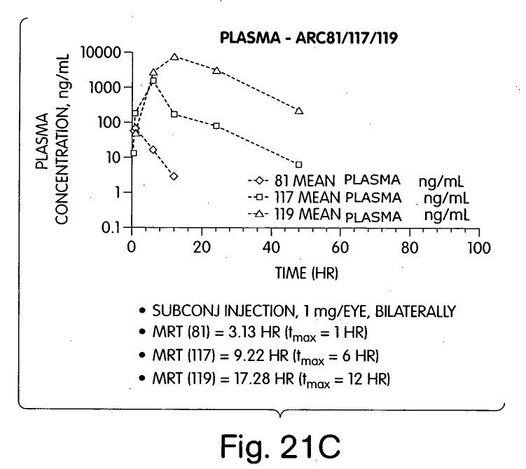

A pharmacokinetic study was performed to determine the pharmacokinetics of ARC81, ARC117 and ARC119, TGFβ2 aptamers having the sequences of SEQ ID NOs:4, 9 and 11, respectively), via subconjunctival administration in mice. These aptamers were formulated at a concentration of 10 mg/ml. Samples were collected at 0, 0.5, 1, 2, 6, 12, 24, 48 and 96 hours post-administration. FIGS. 21A, 21B and 21C show the aqueous humor and/or plasma concentration of each aptamer (in nM) through 50 hours post dose via subconjunctival administration. Table 9 below shows the results of the study at a dose of 1 mg/eye bilaterally (i.e., 2.0 mg/animal).

TABLE 9
Pharmacokinetic profile of ARC81, ARC117 and ARC119 via
subconjunctival administration in mice at 1 mg/eye, bilaterally.

| | | ARC081 | | ARC117 | | ARC119 | |
|---|---|---|---|---|---|---|---|
| | | | Aq | | Aq | | Aq |
| PARAMETER | NITS | PLASMA | Humour | PLASMA | Humour | PLASMA | Humour |
| Cₘₐₓ | nM | 6.44 | 5.19 | 144.8 | 22.9 | 721.1 | 28.4 |
| tₘₐₓ | h | 1 | 6 | 6 | 1 | 12 | 0.5 |
| TLAST | h | 12 | 12 | 48 | 24 | 48 | 48 |
| AUC (0-t) | nM · h | 29.8 | 36.0 | 1,122.1 | 87.6 | 13,380.5 | 133.2 |
| MRT | h | 3.13 | 6.03* | 9.22 | 3.62 | 17.28 | 12.80 |
| t_{1/2} | h | 2.42 | — | 7.40 | 4.33 | 6.94 | 17.52 |
| V_{d} | mL | 21079 | — | 1760 | — | 137 | — |

ARC81, 117 and 119 were detected in the aqueous humor after subconjunctival administration. UnPEGylated aptamers, i.e., ARC81, entered systemic circulation rapidly (e.g., less than 0.5 hour). The Aqueous concentration of unPEGylated aptamer shows delayed tₘₐₓ, possibly due to recirculation of aptamers. The PEGylated aptamers, i.e., ARC117 and ARC119, showed show similar plasma t_{1/2}. The evidence of decreasing V_{d} with PEGylation and delayed tₘₐₓ imply strong depot effect in vicinity of injection site. The PEGylated aptamers were both detected in the aqueous humor and were found to be depoted at the surgical site.

### Example 12

### Comparison of Pharmacokinetic and Bioactivity Profiles of ARC126, ARC127 and NX1838

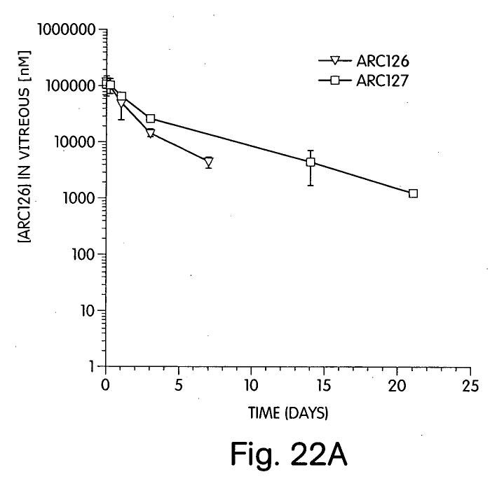

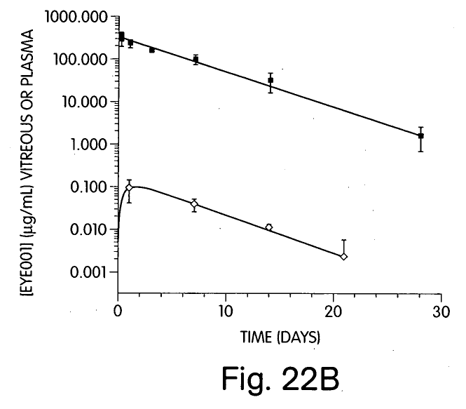

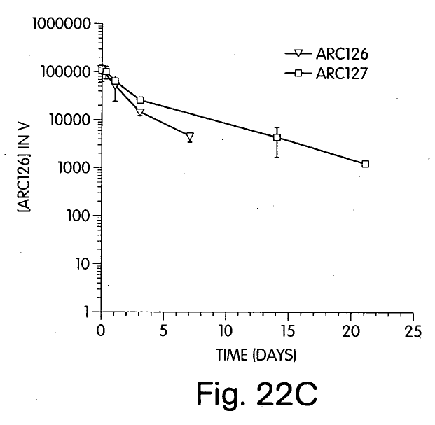

A pharmacokinetic study was performed to determine and compare the pharmacokinetics of ARC126 (SEQ ID NO:18-PEG-SEQ ID NO:33-PEG-SEQ ID NO:34) and ARC127 (PEG-SEQ ID NO:19-PEG-SEQ ID NO:35-PEG-SEQ ID NO:36-3T), two PDGF aptamers, and NX1838, a known PEG-conjugated aptamer directed against VEGF-165 in rabbits. Studies were performed in the Dutch-belted rabbit model using subconjunctival injection. Aptamers were administered at a dose level of 1.0 mg/eye, bilaterally via 100 mL intravitreal injection. Samples from the aqueous humor, vitreous humor and plasma were taken before administration of the aptamer and a 0.25 h, 6 h, 24 h, 72 h, 7d, 4 d, and 21d. FIGS. 22A, 22B and 22C show the vitreous humor and/or plasma concentration of each aptamer through 25 days post administration. Table 10 below shows the results of the study for the ARC126 and ARC127 aptamers.

TABLE 10
Pharmacokinetic profile of ARC126 and ARC127

| | Unit | ARC126 | ARC127 |
|---|---|---|---|
| Cₘₐₓ | μM | 117.91 | 109.18 |
| tₘₐₓ | h | 0.25 | 0.25 |
| AUC_{?} | μM?h | 4,545.2 | 8,122.2 |
| MRT | d | 2.39 | 4.85 |
| t_{1/2} | d | 2.25 | 3.87 |
| Cl | mL?d⁻¹ | 0.52 | 0.29 |
| Vₛₛ | mL | 1.25 | 1.42 |

The results shown in FIG. 22A were obtained using noncompartmental (NCA) analysis. The volume of rabbit vitreous humor was approximately 1.0-1.5 ml. In comparison to the ARC126 and ARC127 aptamers, the half life of NX 1838 in rabbit vitreous was known to be approximately 83 hours, i.e., 3.46 days, and the half life of NX1838 in primate vitreous was known to be approximately 94 hours, or 3.92 days.

As seen in FIGS. 22B and 22C, the Cₘₐₓ (vitreous) was approximately 100 μM for ARC127. Cmax(vit) approximately 100 mm for both nonpegylated and pegylated aptamer conjugates. The C(vit) for pegylated aptamers was approximately 250 nM at t=30 days (40K PEG). The ration of AUC values for the pegylated aptamers and nonpegylated aptamers was 1.79. The half life of nonpegylated aptamers was approximately 2.25 days, and the half life of the pegylated aptamers was approximately 3.87 days. The apparent volume of distribution (Vss) was between 1.25-1.42 mL, which indicates that both conjugates remained in the vitreous compartment. The clearance value (Cl) was between 0.29-0.52 mL/·d (≦50 nmol/day), and the maximum plasma levels were ≦10 nM. The aqueous concentration of the nonpegylated aptamer conjugates was at nanomolar levels for t≦24 hours.

What is claimed is:

1. A pharmaceutical composition for treatment of a disorder of the eye comprising an aptamer which binds specifically to a target involved in said disorder, wherein the binding of the aptamer to the target substantially reduces the effect of the target.

2. The composition of claim 1, wherein said disorder is a cell proliferation disorder.

3. The composition of claim 1, wherein said disorder is characterized by increased intraocular pressure.

4. The composition of claim 3, wherein said disorder is glaucoma.

5. The composition of claim 1, wherein said disorder is post-surgical scarring.

6. The composition of claim 1, wherein said target is selected from the group consisting of cytokines, growth factors, and cell surface proteins.

7. The composition of claim 6, wherein said target is selected from the group consisting of transforming growth factor beta, platelet-derived growth factor, intracellular adhesion molecule-1, insulin-like growth factor-1, vascular endothelial growth factor, tumor necrosis factor alpha, and integrin alpha 5 beta 3.

8. The composition of claim 6, wherein said target is transforming growth factor beta 1, 2, or 3.

9. The composition of claim 8, wherein said transforming growth factor beta is transforming growth factor beta 2.

10. The composition of claim 6, wherein said target is platelet-derived growth factor.

11. The composition of claim 1, further comprising a non-aptamer pharmaceutical agent.

12. The composition of claim 11, wherein the non-aptamer pharmaceutical agent is selected from the group consisting of an anesthetic agent, an anti-inflammatory agent, an anti-angiogenesis agent, an anti-proliferative agent, an anti-bacterial agent, an anti-viral agent, and an anti-fungal agent.

13. The composition of claim 1, further comprising a second aptamer which binds specifically to a target involved in said disorder, wherein the binding of the second aptamer to the target substantially reduces the effect of the target.

14. The composition of claim 13, wherein the first and second aptamers bind specifically to the same type of target involved in said disorder.

15. The composition of claim 13, wherein the first and second aptamers bind specifically to different types of targets involved in said disorder.

16. The composition of claim 1, wherein the aptamer binds specifically to more than one type of target involved in said disorder.

17. The composition of claim 8, comprising an aptamer selected from the group consisting of SEQ ID NOs 1-14, 21-27, 39-149 and 150.

18. The composition of claim 8, comprising an aptamer selected from the group consisting of ARC77, ARC78, ARC81, and ARC154.

19. The composition of claim 10, comprising an aptamer selected from the group consisting of SEQ ID NOs 15, 16 and 17.

20. The composition of claim 10, comprising an aptamer selected from the group consisting of ARC123, ARC124, ARC125, ARC126, ARC127, and ARC128.

21. An aptamer therapeutic for treatment of diseases of the eye, said aptamer having binding specificity to transforming growth factor beta 2 (TGFβ2), wherein said binding of the aptamer to the TGFβ2 substantially reduces the effect of TGFβ2 in cell proliferation in eye disease states.

22. An aptamer therapeutic for treatment of diseases of the eye, said aptamer having binding specificity to transforming growth factor beta 2 (TGFβ2), wherein said binding of the aptamer to the TGFβ2 substantially reduces the effect of TGFβ2 in post-surgical scarring.

23. An aptamer therapeutic for treatment of diseases of the eye, said aptamer having binding specificity to platelet-derived growth factor, wherein said binding of the aptamer to the platelet-derived growth factor substantially reduces the effect of platelet-derived growth factor in cell proliferation in eye disease states.

24. An aptamer therapeutic for treatment of diseases of the eye, said aptamer having binding specificity to platelet-derived growth factor, wherein said binding of the aptamer to the platelet-derived growth factor substantially reduces the effect of platelet-derived growth factor in post-surgical scarring.

25. A method of treating a cell proliferation disorder of the eye comprising the step of administering to a patient a therapeutically effective amount of an aptamer therapeutic, said aptamer having binding specificity to a target involved in said disorder, wherein said binding of the aptamer to the target substantially reduces the effect of the target in cell proliferation in the eye disorder.

26. The method of claim 25, wherein said target is selected from the group consisting of cytokines, growth factors, and cell surface proteins.

27. The method of claim 26, wherein said target is selected from the group consisting of transforming growth factor beta, platelet-derived growth factor, intracellular adhesion molecule-1, insulin-like growth factor-1, vascular endothelial growth factor, tumor necrosis factor alpha, and integrin alpha 5 beta 3.

28. The method of claim 25, wherein said aptamer therapeutic is administered to the ocular cavity.

29. The method of claim 25, wherein said aptamer therapeutic is administered by intravitreal injection.

30. The method of claim 25, wherein said aptamer therapeutic is administered by subconjunctival injection.

31. The method of claim 25, wherein said aptamer therapeutic is administered topically.

32. The composition of claim 1 wherein said aptamer has been modified to increase its stability in aqueous humor present in the eye.

33. The composition of claim 32, wherein said aptamer comprises modified nucleotides.

34. The composition of claim 32, wherein said aptamer comprises a polyalkylene glycol moiety.

35. The composition of claim 34, wherein the polyalkylene glycol moiety is a polyethylene glycol.

36. The composition of claim 34, wherein said aptamer further comprises modified nucleotides.

37. The composition of claim 13, wherein the first and second aptamers are linked by a polyethylene glycol moiety, and further wherein the primary structure of the aptamer composition comprises a linear arrangement in which the first aptamer is linked to a first terminus of the PEG linking moiety and the second aptamer is linked to a second terminus of the PEG linking moiety.

38. The composition of claim 37, wherein the first aptamer is further linked to a terminal polyethylene glycol moiety, wherein the primary structure of the aptamer composition comprises a linear arrangement of polyethylene glycol- first aptamer- polyethylene glycol-second aptamer.

39. An aptamer composition comprising a sequence selected from the group consisting of SEQ ID NO:1-27, 33-150 and 151.


SEQUENCE LISTING (154 sequences)

SEQ ID NO: 1 - RNA, 34 nt, Artificial
  synthetic
ggagguuauuacagagucuguauagcuguacucc

SEQ ID NO: 2 - RNA, 34 nt, Artificial; identical to SEQ ID NO: 1
  synthetic

SEQ ID NO: 3 - RNA, 34 nt, Artificial; identical to SEQ ID NO: 1
  synthetic

SEQ ID NO: 4 - RNA, 32 nt, Artificial
  synthetic
gggguuauuacagagucuguauagcuguaccc

SEQ ID NO: 5 - RNA, 32 nt, Artificial; identical to SEQ ID NO: 4
  synthetic

SEQ ID NO: 6 - RNA, 34 nt, Artificial; identical to SEQ ID NO: 1
  synthetic

SEQ ID NO: 7 - RNA, 34 nt, Artificial; identical to SEQ ID NO: 1
  synthetic

SEQ ID NO: 8 - RNA, 34 nt, Artificial; identical to SEQ ID NO: 1
  synthetic

SEQ ID NO: 9 - RNA, 32 nt, Artificial; identical to SEQ ID NO: 4
  synthetic

SEQ ID NO: 10 - RNA, 32 nt, Artificial; identical to SEQ ID NO: 4
  synthetic

SEQ ID NO: 11 - RNA, 32 nt, Artificial; identical to SEQ ID NO: 4
  synthetic

SEQ ID NO: 12 - RNA, 32 nt, Artificial; identical to SEQ ID NO: 4
  synthetic

SEQ ID NO: 13 - RNA, 32 nt, Artificial; identical to SEQ ID NO: 4
  synthetic

SEQ ID NO: 14 - RNA, 32 nt, Artificial; identical to SEQ ID NO: 4
  synthetic

SEQ ID NO: 15 - DNA, 37 bp, Artificial
  synthetic
tgggagggcgcgttcttcgtggttacttttagtcccg

SEQ ID NO: 16 - DNA, 39 bp, Artificial
  synthetic
cacaggctacggcacgtagagcatcaccatgatcctgtg

SEQ ID NO: 17 - DNA, 44 bp, Artificial
  synthetic
tactcagggcactgcaagcaattgtggtcccaatgggctgagta

SEQ ID NO: 18 - DNA, 9 bp, Artificial
  synthetic
caggcuacg

SEQ ID NO: 19 - DNA, 9 bp, Artificial
  synthetic
caggcuacg

SEQ ID NO: 20 - DNA, 9 bp, Artificial
  synthetic
cagcguacg

SEQ ID NO: 21 - RNA, 34 nt, Artificial; identical to SEQ ID NO: 1
  synthetic

SEQ ID NO: 22 - RNA, 34 nt, Artificial; identical to SEQ ID NO: 1
  synthetic

SEQ ID NO: 23 - RNA, 32 nt, Artificial; identical to SEQ ID NO: 4
  synthetic

SEQ ID NO: 24 - RNA, 32 nt, Artificial; identical to SEQ ID NO: 4
  synthetic

SEQ ID NO: 25 - RNA, 32 nt, Artificial; identical to SEQ ID NO: 4
  synthetic

SEQ ID NO: 26 - RNA, 32 nt, Artificial; identical to SEQ ID NO: 4
  synthetic

SEQ ID NO: 27 - RNA, 34 nt, Artificial; identical to SEQ ID NO: 1
  synthetic

SEQ ID NO: 28 - DNA, 85 bp, Artificial
  synthetic
tcgggcgagtcgtctggaaggaattttactacaacgttacttccgcatcctccccgcatcgtcctccctatagtgagtcgtatta

SEQ ID NO: 29 - DNA, 85 bp, Artificial
  synthetic
tcgggcgagtcgtctggcggacttagtatatacatacgactaaacaacgccgcccgcatcgtcctccctatagtgagtcgtatta

SEQ ID NO: 30 - DNA, 82 bp, Artificial
  synthetic
tcgggcgagtcgtctgggagtacagctatacagactctgtaataacctccccgcatcgtcctccctatagtgagtcgtatta

SEQ ID NO: 31 - DNA, 32 bp, Artificial
  synthetic
taatacgactcactatagggaggacgatgcgg

SEQ ID NO: 32 - DNA, 16 bp, Artificial
  synthetic
tcgggcgagtcgtctg

SEQ ID NO: 33 - DNA, 12 bp, Artificial
  synthetic
cgtagagcauca

SEQ ID NO: 34 - DNA, 8 bp, Artificial
  synthetic
tgatccug

SEQ ID NO: 35 - DNA, 12 bp, Artificial
  synthetic
cgtagagcauca

SEQ ID NO: 36 - DNA, 8 bp, Artificial
  synthetic
tgatccug

SEQ ID NO: 37 - DNA, 12 bp, Artificial
  synthetic
cgtaccgatuca

SEQ ID NO: 38 - DNA, 8 bp, Artificial
  synthetic
tgaagcug

SEQ ID NO: 39 - RNA, 65 nt, Artificial
  synthetic
gggaggacgaugcggaucgaguauuauagaguauguauagcuauacgaucagacgacucgcccga

SEQ ID NO: 40 - RNA, 65 nt, Artificial
  synthetic
gggaggacgaugcggaucgaguauuauagaguauguauagcuauacuaucagacgacucgcccga

SEQ ID NO: 41 - RNA, 65 nt, Artificial
  synthetic
gggaggacgaugcggaucgaauauuauagaguauguauagcuauacggucagacgacucgcccga

SEQ ID NO: 42 - RNA, 65 nt, Artificial
  synthetic
gggaggacgauggggaucgaguauuauagagucuguauagcuauacgaucagacgacucgcccga

SEQ ID NO: 43 - RNA, 65 nt, Artificial
  synthetic
gggaggacgaugcggauggaguauuauagaguauguauagcuauaccaucagacgacucgcccga

SEQ ID NO: 44 - RNA, 65 nt, Artificial
  synthetic
gggaggacgaugcggauagagcauuauagaguauguauagcuauacuaucagacgacucgcccga

SEQ ID NO: 45 - RNA, 65 nt, Artificial
  synthetic
gggaggacgaugcggauagaguauuauagaguauguauagcuauacuaucagacgacucgcccga

SEQ ID NO: 46 - RNA, 65 nt, Artificial
  synthetic
gggaggacgaugcggacagaguauuauagaguauguguagcuauaccgucagacgacucgcccga

SEQ ID NO: 47 - RNA, 65 nt, Artificial
  synthetic
gggaggacgaugcggacagaguauuauagaguauguauagcuauacugccagacgacucgcccga

SEQ ID NO: 48 - RNA, 64 nt, Artificial
  synthetic
gggaggacgaugcggacagaguauuauagaguauguauagcuauacugcagacgacucgcccga

SEQ ID NO: 49 - RNA, 65 nt, Artificial
  synthetic
gggaggacgaugcggacagagcauuauagaguguguauagcuguacugucagacgacucgcccga

SEQ ID NO: 50 - RNA, 65 nt, Artificial
  synthetic
gggaggacgaugcggacagaguauuauagaguauguauagcuauacuaucagacgacucgcccaa

SEQ ID NO: 51 - RNA, 66 nt, Artificial
  synthetic
gggaggacgaugcggacagaguauuauagaguauguauagcuauacuguccagacgacucgcccga

SEQ ID NO: 52 - RNA, 65 nt, Artificial
  synthetic
gggaggacgaugcggacagaguauuauagaguauguauagcuauacugucagacgacucgcccga

SEQ ID NO: 53 - RNA, 65 nt, Artificial
  synthetic
gggaggacgaugcggaaagaguauuauagagucuguauagcuauacuuucagacgacucgcccga

SEQ ID NO: 54 - RNA, 65 nt, Artificial
  synthetic
gggaggacgaugcggaangaauauuauagaguauguauagcuauaccaucagacgacucgcccga

SEQ ID NO: 55 - RNA, 62 nt, Artificial
  synthetic
gggaggacgaugcggaaggaguauuauagaguauguauagcuauaccagacgacucgcccga

SEQ ID NO: 56 - RNA, 65 nt, Artificial
  synthetic
gggaggacgaugcggaaggaguauuauagaguauguauagcuauaccaucagacgacucgcccga

SEQ ID NO: 57 - RNA, 65 nt, Artificial
  synthetic
gggaggacgaugcggaaggaauauuauagaguauguauagcuauaccaucagacgacucgcccga

SEQ ID NO: 58 - RNA, 65 nt, Artificial
  synthetic
gggaggacgaugcggagggauuauuauagagucuguauagcuauacccucagacgacucgcccga

SEQ ID NO: 59 - RNA, 65 nt, Artificial
  synthetic
gggaggacgaugcggagggaauauuauagaguauguauagcuauaccaucagacgacucgcccga

SEQ ID NO: 60 - RNA, 65 nt, Artificial
  synthetic
gggaggacgaugcggagaagaguauuauagagucuguauagcuauacuucagacgacucgcccga

SEQ ID NO: 61 - RNA, 65 nt, Artificial
  synthetic
gggaggacgaugcggagagauuauuauagaguauguauagcuguacugccagacgacucgcccga

SEQ ID NO: 62 - RNA, 65 nt, Artificial
  synthetic
gggaggacgaugcggaacgaauauuacagaguauguauagcuguacggucagacgacucgcccga

SEQ ID NO: 63 - RNA, 66 nt, Artificial
  synthetic
gggaggacgaugcggagugaguauuauagaguauguauagcuauacacaucagacgacucgcccga

SEQ ID NO: 64 - RNA, 65 nt, Artificial
  synthetic
gggaggacgaugcggugugaauauuauagagucuguauagcuauaccaucagacgacucgcccga

SEQ ID NO: 65 - RNA, 65 nt, Artificial
  synthetic
gggaggacgaugcggugugaauauuauagagucuguauagcuauaccaccagacgacucgcccga

SEQ ID NO: 66 - RNA, 65 nt, Artificial
  synthetic
gggaggacgaugcggugugaguauuauagagucuguauagcuauaccaccagacgacucgcccga

SEQ ID NO: 67 - RNA, 68 nt, Artificial
  synthetic
gggaggacgaugcgguccgcauuaucuucuacguuacauauacuaucucugucagacgacucgcccga

SEQ ID NO: 68 - RNA, 65 nt, Artificial
  synthetic
gggaggacgaugcggucggaguauuauagaguauguauagcuaaaccgucagacgacucgcccga

SEQ ID NO: 69 - RNA, 66 nt, Artificial
  synthetic
gggaggacgaugcgguaagaguauuacagaguauguauagcuguacuugccagacgacucgcccga

SEQ ID NO: 70 - RNA, 66 nt, Artificial
  synthetic
gggaggacgaugcggugagaauauuacagaguauguauagcuguacuugccagacgacucgcccga

SEQ ID NO: 71 - RNA, 66 nt, Artificial
  synthetic
gggaggacgaugcggugagaauauuauagaguauguauagcuauacucgccagacgacucgcccaa

SEQ ID NO: 72 - RNA, 65 nt, Artificial
  synthetic
gggaggangangcggugagaguauuauagagucuguauagcuauacugccagacgacucgcccga

SEQ ID NO: 73 - RNA, 66 nt, Artificial
  synthetic
gggaggacgaugcggugagaguauuauagagucuguauagcuauacucgccagacgacucgcccga

SEQ ID NO: 74 - RNA, 65 nt, Artificial
  synthetic
gggaggacgaugcggugagaguauuauagagucuguauagcuauaccaccagacgacucgcccga

SEQ ID NO: 75 - RNA, 64 nt, Artificial
  synthetic
gggaggacgaugcgguagaguauuauagaguauguauagcuauacacucagacgacucgcccaa

SEQ ID NO: 76 - RNA, 64 nt, Artificial
  synthetic
gggaggacgaugcgguagaguauuauagaguauguauagcuauaccaucagacgacucgcccga

SEQ ID NO: 77 - RNA, 65 nt, Artificial
  synthetic
gggaggacgaugcggugggaauauuauagagucuguauagcuauacccucagacgacucgcccga

SEQ ID NO: 78 - RNA, 65 nt, Artificial
  synthetic
gggaggacgaugcgggcggaauauuauagaguauggauagcuauaccgucagacgacucgcccga

SEQ ID NO: 79 - RNA, 66 nt, Artificial
  synthetic
gggaggacgaugcgggacagaguauuauagaguauguauagcuauacugucagacgacucgcccga

SEQ ID NO: 80 - RNA, 65 nt, Artificial
  synthetic
gggaggacgaugcgggcagaguauuauagaguacguauagcuauacugucagacgacucgcccga

SEQ ID NO: 81 - RNA, 65 nt, Artificial
  synthetic
gggaggacgaugcgggcagaguauuauagaguauguauagcuauacugucagacgacucgcccga

SEQ ID NO: 82 - RNA, 65 nt, Artificial
  synthetic
gggaggacgaugcggguggaguauuauagaguauguauagcuauacuaucagacgacucgcccaa

SEQ ID NO: 83 - RNA, 65 nt, Artificial
  synthetic
gggaggacgaugcggguagauuauuacagagucuguauagcuguacugccagacgacucgcccga

SEQ ID NO: 84 - RNA, 65 nt, Artificial
  synthetic
gggaggacgaugcggguagaauauuauagagucuguauagcuauacugccagacgacucgcccga

SEQ ID NO: 85 - RNA, 65 nt, Artificial
  synthetic
gggaggacgaugcggguagaauauuacagaguauguauagcuguacugccagacgacucgcccga

SEQ ID NO: 86 - RNA, 66 nt, Artificial
  synthetic
gggaggacgaugcggggagaauauuacagaguauguauagcuguacuugccagacgacucgcccga

SEQ ID NO: 87 - RNA, 65 nt, Artificial
  synthetic
gggaggacgaugcggggagaauauuacagaguauguauagcuguacugccagacgacucgcccga

SEQ ID NO: 88 - RNA, 65 nt, Artificial
  synthetic
gggaggacgaugcggggagauuauuauagaguauguauagcuauacugccagacgacucgcccga

SEQ ID NO: 89 - RNA, 65 nt, Artificial
  synthetic
gggaggacgaugcggggagaguauuauagaguauguauagcuauacugccagacgacucgcccga

SEQ ID NO: 90 - RNA, 65 nt, Artificial
  synthetic
gggaggacgaugcgggaagaguauuacagaguauguauagcuguacugccagacgacucgcccga

SEQ ID NO: 91 - RNA, 65 nt, Artificial
  synthetic
gggaggacgaugcgggcaaaguauuguagaguaugcauagcuauauugccagacgacucgcccga

SEQ ID NO: 92 - RNA, 65 nt, Artificial
  synthetic
gggaggacgaugcggggagguuauuacagagucuguauagcuguacucccagacgacucgcccga

SEQ ID NO: 93 - RNA, 68 nt, Artificial
  synthetic
gggaggacgaugcggggaggaugcggaaguaacguuguaguaaaauuccuuccagacgacucgcccga

SEQ ID NO: 94 - RNA, 68 nt, Artificial
  synthetic
gggaggacgaugcgggcggcguuguuuagucguauguauauacuaaguccgccagacgacucgcccga

SEQ ID NO: 95 - RNA, 34 nt, Artificial
  synthetic
aucgaguauuauagaguauguauagcuauacgau

SEQ ID NO: 96 - RNA, 34 nt, Artificial
  synthetic
aucgaguauuauagaguauguauagcuauacuau

SEQ ID NO: 97 - RNA, 34 nt, Artificial
  synthetic
aucgaauauuauagaguauguauagcuauacggu

SEQ ID NO: 98 - RNA, 34 nt, Artificial
  synthetic
aucgaguauuauagagucuguauagcuauacgau

SEQ ID NO: 99 - RNA, 34 nt, Artificial
  synthetic
auggaguauuauagaguauguauagcuauaccau

SEQ ID NO: 100 - RNA, 34 nt, Artificial
  synthetic
auagagcauuauagaguauguauagcuauacuau

SEQ ID NO: 101 - RNA, 34 nt, Artificial
  synthetic
auagaguauuauagaguauguauagcuauacuau

SEQ ID NO: 102 - RNA, 34 nt, Artificial
  synthetic
acagaguauuauagaguauguguagcuauaccgu

SEQ ID NO: 103 - RNA, 34 nt, Artificial
  synthetic
acagaguauuauagaguauguauagcuauacugc

SEQ ID NO: 104 - RNA, 33 nt, Artificial
  synthetic
acagaguauuauagaguauguauagcuauacug

SEQ ID NO: 105 - RNA, 34 nt, Artificial
  synthetic
acagagcauuauagaguguguauagcuguacugu

SEQ ID NO: 106 - RNA, 34 nt, Artificial
  synthetic
acagaguauuauagaguauguauagcuauacuau

SEQ ID NO: 107 - RNA, 35 nt, Artificial
  synthetic
acagaguauuauagaguauguauagcuauacuguc

SEQ ID NO: 108 - RNA, 34 nt, Artificial
  synthetic
acagaguauuauagaguauguauagcuauacugu

SEQ ID NO: 109 - RNA, 34 nt, Artificial
  synthetic
aaagaguauuauagagucuguauagcuauacuuu

SEQ ID NO: 110 - RNA, 34 nt, Artificial
  synthetic
aangaauauuauagaguauguauagcuauaccau

SEQ ID NO: 111 - RNA, 31 nt, Artificial
  synthetic
aaggaguauuauagaguauguauagcuauac

SEQ ID NO: 112 - RNA, 34 nt, Artificial
  synthetic
aaggaguauuauagaguauguauagcuauaccau

SEQ ID NO: 113 - RNA, 34 nt, Artificial
  synthetic
aaggaauauuauagaguauguauagcuauaccau

SEQ ID NO: 114 - RNA, 34 nt, Artificial
  synthetic
agggauuauuauagagucuguauagcuauacccu

SEQ ID NO: 115 - RNA, 34 nt, Artificial
  synthetic
agggaauauuauagaguauguauagcuauaccau

SEQ ID NO: 116 - RNA, 34 nt, Artificial
  synthetic
agaagaguauuauagagucuguauagcuauacuu

SEQ ID NO: 117 - RNA, 34 nt, Artificial
  synthetic
agagauuauuauagaguauguauagcuguacugc

SEQ ID NO: 118 - RNA, 34 nt, Artificial
  synthetic
aacgaauauuacagaguauguauagcuguacggu

SEQ ID NO: 119 - RNA, 35 nt, Artificial
  synthetic
agugaguauuauagaguauguauagcuauacacau

SEQ ID NO: 120 - RNA, 34 nt, Artificial
  synthetic
ugugaauauuauagagucuguauagcuauaccau

SEQ ID NO: 121 - RNA, 34 nt, Artificial
  synthetic
ugugaauauuauagagucuguauagcuauaccac

SEQ ID NO: 122 - RNA, 34 nt, Artificial
  synthetic
ugugaguauuauagagucuguauagcuauaccac

SEQ ID NO: 123 - RNA, 37 nt, Artificial
  synthetic
uccgcauuaucuucuacguuacauauacuaucucugu

SEQ ID NO: 124 - RNA, 34 nt, Artificial
  synthetic
ucggaguauuauagaguauguauagcuaaaccgu

SEQ ID NO: 125 - RNA, 35 nt, Artificial
  synthetic
uaagaguauuacagaguauguauagcuguacuugc

SEQ ID NO: 126 - RNA, 35 nt, Artificial
  synthetic
ugagaauauuacagaguauguauagcuguacuugc

SEQ ID NO: 127 - RNA, 35 nt, Artificial
  synthetic
ugagaauauuauagaguauguauagcuauacucgc

SEQ ID NO: 128 - RNA, 34 nt, Artificial
  synthetic
ugagaguauuauagagucuguauagcuauacugc

SEQ ID NO: 129 - RNA, 35 nt, Artificial
  synthetic
ugagaguauuauagagucuguauagcuauacucgc

SEQ ID NO: 130 - RNA, 34 nt, Artificial
  synthetic
ugagaguauuauagagucuguauagcuauaccac

SEQ ID NO: 131 - RNA, 33 nt, Artificial
  synthetic
uagaguauuauagaguauguauagcuauacacu

SEQ ID NO: 132 - RNA, 33 nt, Artificial
  synthetic
uagaguauuauagaguauguauagcuauaccau

SEQ ID NO: 133 - RNA, 30 nt, Artificial
  synthetic
aauauuauagagucuguauagcuauacccu

SEQ ID NO: 134 - RNA, 34 nt, Artificial
  synthetic
gcggaauauuauagaguauggauagcuauaccgu

SEQ ID NO: 135 - RNA, 35 nt, Artificial
  synthetic
gacagaguauuauagaguauguauagcuauacugu

SEQ ID NO: 136 - RNA, 34 nt, Artificial
  synthetic
gcagaguauuauagaguacguauagcuauacugu

SEQ ID NO: 137 - RNA, 34 nt, Artificial
  synthetic
gcagaguauuauagaguauguauagcuauacugu

SEQ ID NO: 138 - RNA, 34 nt, Artificial
  synthetic
guggaguauuauagaguauguauagcuauacuau

SEQ ID NO: 139 - RNA, 34 nt, Artificial
  synthetic
guagauuauuacagagucuguauagcuguacugc

SEQ ID NO: 140 - RNA, 34 nt, Artificial
  synthetic
guagaauauuauagagucuguauagcuauacugc

SEQ ID NO: 141 - RNA, 34 nt, Artificial
  synthetic
guagaauauuacagaguauguauagcuguacugc

SEQ ID NO: 142 - RNA, 35 nt, Artificial
  synthetic
ggagaauauuacagaguauguauagcuguacuugc

SEQ ID NO: 143 - RNA, 34 nt, Artificial
  synthetic
ggagaauauuacagaguauguauagcuguacugc

SEQ ID NO: 144 - RNA, 34 nt, Artificial
  synthetic
ggagauuauuauagaguauguauagcuauacugc

SEQ ID NO: 145 - RNA, 34 nt, Artificial
  synthetic
ggagaguauuauagaguauguauagcuauacugc

SEQ ID NO: 146 - RNA, 34 nt, Artificial
  synthetic
gaagaguauuacagaguauguauagcuguacugc

SEQ ID NO: 147 - RNA, 34 nt, Artificial
  synthetic
gcaaaguauuguagaguaugcauagcuauauugc

SEQ ID NO: 148 - RNA, 34 nt, Artificial; identical to SEQ ID NO: 1
  synthetic

SEQ ID NO: 149 - RNA, 37 nt, Artificial
  synthetic
ggaggaugcggaaguaacguuguaguaaaauuccuuc

SEQ ID NO: 150 - RNA, 37 nt, Artificial
  synthetic
gcggcguuguuuagucguauguauauacuaaguccgc

SEQ ID NO: 151 - RNA, 32 nt, Artificial
  synthetic
ggaguuauacagagucuguauagcuguacucc

SEQ ID NO: 152 - protein, 112 aa, Homo sapiens
ALDAAYCFRNVQDNCCLRPLYIDFKRDLGWKWIHEPKGYNANFCAGACPLYLWSSDTQHSRVLSLYNTINPEASASPCCVSQDLEPLTILYYIGKTKIEQLSNMIVKSCKCS

SEQ ID NO: 153 - protein, 146 aa, Homo sapiens
MRGSHHHHHHGMASMTGGQQMGRDLYDDDDKDRWALDAAYCFRNVQDNCCLRPLYIDFKRDLGWKWIHEPKGYNANFCAGACPLYLWSSDTQHSRVLSLYNTINPEASASPCCVSQDLEPLTILYYIGKTKIEQLSNMIVKSCKCS

SEQ ID NO: 154 - protein, 118 aa, Homo sapiens
MHHHHHALDAAYCFRNVQDNCCLRPLYIDFKRDLGWKWIHEPKGYNANFCAGACPLYLWSSDTQHSRVLSLYNTINPEASASPCCVSQDLEPLTILYYIGKTKIEQLSNMIVKSCKCS